(19) United States
(12) Patent Application Publication
(10) Pub. No.: US 2023/0365555 A1
(43) Pub. Date: Nov. 16, 2023

(54) CRYSTALLINE POLYMORPH FORM A OF A JAK INHIBITORAND METHODS FOR ITS PREPARATION

(71) Applicant: ACLARIS THERAPEUTICS, INC., Wayne, PA (US)

(72) Inventors: Gary DECRESCENZO, Parkville, MO (US); John R. SPRINGER, Wentzville, MO (US); Jon P, LAWSON, Wildwood, MO (US)

(21) Appl. No.: 18/028,157

(22) PCT Filed: Sep. 24, 2021
(86) PCT No.: PCT/US2021/052063

§ 371 Date: Mar. 31, 2023

Related U.S. Application Data
(60) Provisional application No. 63/083,663, filed on Sep. 25, 2020.

(51) Int. Cl. C07D 471/04

(52) U.S. Cl. CPC C07D 471/04; C07B 2200/13

(57) ABSTRACT

The present application relates to a novel crystalline form of ethyl (R)-4-((1-(2-cyanoacetyl)piperidin-3-yl)amino)-1H-pyrrolo[2,3 -b]pyridine-5-carboxylate (Compound 1). The present application also relates to a method of preparing Compound 1 and a method of preparing crystalline polymorph Form A of Compound 1. Additional aspects of the present application relate to pharmaceutical compositions comprising Compound 1 formed from the crystalline form or the crystalline form of Compound 1.

### CROSS-REFERENCE TO RELATED APPLICATIONS

This application claims the benefit of U.S. Provisional Application No. 63/083,663 filed Sep. 25, 2020, which is hereby incorporated by reference in its entirey.

### SUMMARY OF THE INVENTION

In one aspect, the present disclosure provides a novel crystalline form of ethyl (R)-4-((1-(2-cyanoacetyl)piperidin-3-yl)amino)-1H-pyrrolo[2,3 -b]pyridine-5-carboxylate (Compound 1):

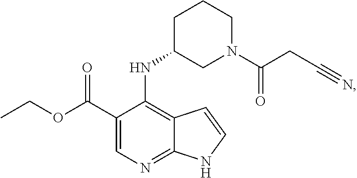 [SMILES: CCOC(=O)c1cnc2[nH]ccc2c1N[C@@H]1CCCN(C(=O)CC#N)C1]

described and identified herein as crystalline polymorph Form A of Compound 1. Crystalline polymorph Form A of Compound 1 may be characterized by a PXRD pattern comprising a significant peak at a 2θ angle of about 10.50°. A PXRD pattern of crystalline polymorph Form A of Compound 1 may additionally comprise a significant peak at 2θ angle of about 18.86°. A PXRD pattern of crystalline polymorph Form A of Compound 1 may additionally comprise significant peaks at 2θ angles of about 9.69°, about 14.01°, and about 25.85°. Yet further, a PXRD pattern of crystalline polymorph Form A of Compound 1 may additionally comprise significant peaks at 2θ angles of about 4.67°, about 9.33°, about 9.55°, and about 27.46°.

Alternatively or in addition to the PXRD pattern, crystalline polymorph Form A of Compound 1 may be characterized by a FT-Raman spectra comprising a significant peak at about 1499.7 cm⁻¹. A FT-Raman spectra of crystalline polymorph Form A of Compound 1 may additionally comprise a significant peak at about 31.867 cm⁻¹. A FT-Raman spectra of crystalline polymorph Form A of Compound 1 may additionally comprise significant peaks at about 28.008 cm⁻¹, about 27.729 cm⁻¹, about 20.742 cm⁻¹, and about 19.862 cm⁻¹. A FT-Raman spectra of crystalline polymorph Form A of Compound 1 may additionally comprise significant peaks at about 17.799 cm⁻¹, about 17.727 cm⁻¹, about 17.47 cm⁻¹, and about 16.713 cm⁻¹.

Crystalline polymorph Form A of Compound 1 may be characterized by a PXRD pattern comprising a significant peak at a 2θ angle of about 10.50° and characterized by a FT-Raman spectra comprising a significant peak at about 1499.7 cm⁻¹. Crystalline polymorph Form A of Compound 1 may be characterized by a PXRD pattern further comprising a significant peak at 2θ angle of about 18.86° and a FT-Raman spectra further comprising a significant peak at about 31.867 cm⁻¹. Crystalline polymorph Form A of Compound 1 may be characterized by a PXRD pattern further comprising significant peaks at 2θ angle of about 9.69°, about 14.01°, and about 25.85° and a FT-Raman spectra further comprising significant peaks at about 28.008 cm⁻¹, about 27.729 cm⁻¹, about 20.742 cm⁻¹, and about 19.862 cm⁻¹. Crystalline polymorph Form A of Compound 1 may be characterized by a PXRD pattern further comprising significant peaks at 2θ angle of about 4.67°, about 9.33°, about 9.55°, and about 27.46° and a FT-Raman spectra further comprising significant peaks at about 17.799 cm⁻¹, about 17.727 cm⁻¹, about 17.47 cm⁻¹, and about 16.713 cm⁻¹.

In various embodiments, crystalline polymorph Form A of Compound 1 may be further characterized by a PXRD pattern substantially as shown in FIG. 2 or FIG. 3. Crystalline polymorph Form A of Compound 1 may also be characterized by one or more of 1) a DSC thermograms exhibiting an endotherm at about 196.8° C.; 2) a water loss as measured by thermogravimetric analysis of about 0.7 wt. %; and 3) an FT-Raman spectra as substantially shown in FIG. 6.

In yet another aspect, the present disclosure provides a method of preparing crystalline polymorph Form A of Compound 1. This method comprises combining Compound 1 and an alcohol to form a reaction mixture; heating the reaction mixture to a temperature sufficient to form a clear solution; and cooling the clear solution to a temperature of about 10° C. to about 15° C. to yield crystalline polymorph Form A of Compound 1 as a solid precipitate. In various embodiments, the reaction mixture may be heated to a temperature of about 70° C. to about 75° C. In various embodiments, the alcohol is ethanol. Cooling the clear solution to a temperature of about 10° C. to about 15° C. may be carried out in a stepwise fashion, for example, by a first cooling of the reaction mixture to a temperature of about 40° C. to about 45° C. over a time period of about 3-4 hours; a second cooling of the reaction mixture to a temperature of about 25° C. to about 30° C. over a period of about 3 hours to about 4 hours; and, finally, a third cooling of the reaction mixture to a temperature of about 10° C. to about 15° C. over a period of about 2 hours to about 3 hours.

At the conclusion of each of the above described steps, the reaction mixture may be maintained at the ending temperature. For example, the reaction mixture may be maintained at a temperature of 40° C. to about 45° C. for about 6 hours to about 7 hours prior to cooling the reaction mixture to a temperature of about 25° C. to about 30° C. Additionally or alternatively, the reaction mixture may be maintained at a temperature of about 25° C. to about 30° C. for about 12 hours to about 14 hours prior to cooling the reaction mixture to a temperature of about 10° C. to about 15° C. Additionally or alternatively, the reaction mixture may be maintained at about 10° C. to about 15° C. for about 4 hours to about 6 hours to yield crystalline polymorph Form A of Compound 1 as a solid precipitate.

Optionally, activated carbon may be added to the clear solution prior to cooling the clear solution to a temperature of about 10° C. to about 15° C. The solution may be mixed and then filtered to remove the activated carbon. Optionally, after isolating crystalline polymorph Form A as a solid, the solid may be further processed, for example, by drying in a vacuum at a temperature of about 50° C. to about 55° C.

In yet another aspect, the present disclosure provides a method of preparing

Compound 1 having the structure:

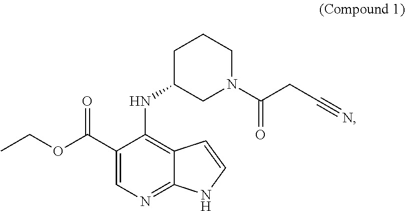 [SMILES: CCOC(=O)c1cnc2[nH]ccc2c1N[C@@H]1CCCN(C(=O)CC#N)C1]

comprising the steps of: (a) contacting the compound

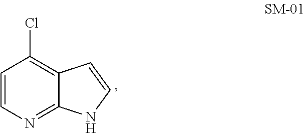

with the compound

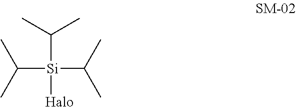

in the presence of a base to form the compound

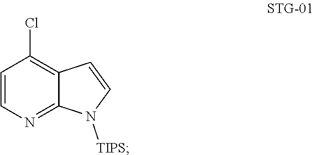

and (b) converting STG-01 to Compound 1.

Yet another aspect of the present invention provides a pharmaceutical composition comprising a therapeutically effective amount of Compound 1 prepared from the crystalline polymorph Form A of Compound 1 or a crystalline polymorph Form A of Compound 1, a pharmaceutically acceptable salt thereof, or a combination thereof; and a pharmaceutically acceptable carrier.

In yet another aspect, the present invention provides a method of treating a JAK1 and/or JAK3-mediated disease in a subject in need thereof comprising administering to the subject a therapeutically effective amount of Compound 1 prepared from the crystalline polymorph Form A of Compound 1 or a crystalline polymorph Form A of Compound 1, or a combination thereof.

### BRIEF DESCRIPTION OF THE DRAWINGS

For a fuller understanding of the nature and advantages of the present invention, reference should be made to the following detailed description taken in connection with the accompanying drawings, in which:

FIG. 1A provides an image of crystalline Compound 1 under optical microscope. FIGS. 1B and 1C provide images of a single crystal used for the single crystal measurements used to simulate a PXRD pattern for Compound 1.

FIG. 2 is a representative PXRD pattern collected from a sample of Compound 1 Polymorph Form A.

FIG. 3 is a PXRD pattern simulated from measurements of a single crystal of Compound 1 Polymorph Form A.

FIG. 4 is a thermogravimetric analysis (TGA) curve and a differential scanning calorimetry (DSC) curve for Compound 1.

FIG. 5 is a dynamic vapor sorption (DVS) isotherms for Compound 1.

FIG. 6 is a FT-Raman spectrum for Compound 1.

### DETAILED DESCRIPTION

Before the present compositions and methods are described, it is to be understood that the scope of the invention is not limited to these particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference with respect to the aspect it is identified as describing. Nothing herein is to be construed as an admission that the invention is not entitled to antedate such disclosure by virtue of prior invention.

Atopic dermatitis (AD) is a common, chronic skin disorder caused by complex genetic, immunological, and environmental interactions. AD has no definitive laboratory or histological findings, however, the hallmark of this condition is a disturbance of epidermal-barrier function due to recurrent skin inflammation, leading to dry skin, pruritus, and IgE-mediated allergen sensitization. AD significantly impairs quality of life for both child and family. Treatment of AD is aimed at continuous epidermal-barrier repair through the use of emollients and avoidance of personal triggering factors, however, use of targeted inhibitors of janus kinases has recently emerged as an effective alternative to conventional therapies.

The following disclosure and description is provided based at least in part on the clinical observation that the topical application of a therapeutically effective amount of a composition comprising ethyl (R)-4-((1-(2-cyanoacetyl)piperidin-3-yl)amino)-1H-pyrrolo[2,3-b]pyridine-5-carboxylate (herein “Compound 1”), an inhibitor of janus kinase 1 (JAK1) and janus kinase 3 (JAK3) to the skin is effective in treating various skin conditions and disorders, e.g., atopic dermatitis, vitiligo, and alopecia areata.

Compound 1, shown below as Formula I, is disclosed in U.S. Patent Application Publication No. 20190135808A1 (Example 117), which is hereby incorporated by reference with respect to its disclosure of and methods of synthesizing Compound 1. Compound 1, in its freebase form, has a chemical formula of C₁₈H₂₁N₅O₃ and a molecular weight of 355.40 g/mol.

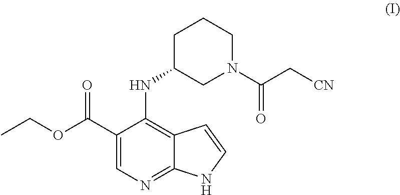 [SMILES: CCOC(=O)c1cnc2[nH]ccc2c1N[C@@H]1CCCN(C(=O)CC#N)C1]

As used herein, “Compound 1” refers to ethyl (R)-4-((1-(2-cyanoacetyl)piperidin-3-yl)amino)-1H-pyrrolo[2,3-b]pyridine-5-carboxylate.

### Definitions

It must also be noted that as used herein and in the appended claims, the singular forms “a”, “an”, and “the” include plural reference unless the context clearly dictates otherwise. Thus, for example, reference to a “JAK inhibitor” is a reference to one or more JAK inhibitors and equivalents thereof known to those skilled in the art, and so forth.

As used herein, the term “about” means plus or minus 10% of the numerical value of the number with which it is being used. Therefore, about 50% means in the range of 45% to 55%. When used to describe temperature, “about” refers to the identified temperature plus or minus 5 degrees. When used to describe PXRD peaks, the term “about” refers to the identified 20 peak plus or minus 0.2 degrees.

In any embodiment, the methods and compositions disclosed herein may comprise the recited steps and components. As used here, “comprise” is open language used to recite steps or components that are included in the recited method or composition but indicate that other elements may also be included, even though said elements are not explicitly recited. In any embodiment, the methods and compositions disclosed herein may consist essentially of the recited steps and components. As used here, “consist essentially of” is used to recite steps or components that are included in the recited method or composition and to indicate that other elements may also be included, but said other elements would not materially affect the properties of the composition or the results of the method. In any embodiment, the methods and compositions disclosed herein may consist of the recited steps and components. As used here, “consist of” is closed language used to recite steps or components that are included in the recited method or composition and that no other elements are included other than those explicitly recited. Any use of the term comprise or comprising may be replaced with “consisting essentially of” or “consisting of.”

As used herein, two embodiments are “mutually exclusive” when one is defined to be something which is different from the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

As used herein, the term “pharmaceutically acceptable salt” refers to a salt prepared from an acid which is acceptable for administration to a patient. The term “pharmaceutically acceptable salts” embraces salts commonly used to form alkali metal salts and to form addition salts from free acids. Such salts can be derived from from pharmaceutically-acceptable inorganic or organic acids.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “halo,” or “halogen,” as used herein, alone or in combination, refers to fluorine, chlorine, bromine, or iodine.

The term “substantially free” as used herein, alone or in combination, refers to a compound which is free from all other compounds within the limits of detection as measured by any means including nuclear magnetic resonance (NMR), gas chromatography/mass spectroscopy (GC/MS), or liquid chromatography/mass spectroscopy (LC/MS).

Stereogenic centers exist in some of the compounds disclosed herein. These centers are designated by the symbols “R” or “S,” depending on the configuration of substituents around the stereogenic center. It should be understood that the invention encompasses all stereochemical isomeric forms, including diastereomeric, enantiomeric, atropisomeric, racemic and epimeric forms, as well as d-isomers and 1-isomers, and mixtures thereof. Individual stereoisomers of compounds can be prepared synthetically from commercially available starting materials which contain fixed stereogenic centers or by preparation of racemic mixtures of products followed by enantiomeric separation such as conversion to a mixture of diastereomers followed by separation or recrystallization, chromatographic techniques, direct separation of enantiomers on chiral chromatographic columns, or any other appropriate method known in the art. Starting compounds of particular stereochemical configuration are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single topical composition having a fixed ratio of active ingredients or in multiple, separate topical compositions for each active ingredient. In addition, such administration also encompasses use of each type of therapeutic agent in a sequential manner. In either case, the treatment regimen will provide beneficial effects of the drug combination in treating the conditions or disorders described herein.

“JAK1 and/or JAK3 inhibitor” is used herein to refer to a compound that exhibits an IC₅₀ with respect to JAK1 and/or JAK3 activity of no more than about 100 μM and more typically not more than about 50 μM, as measured in the JAK1 and JAK3 enzyme assays described generally herein. In some embodiments, the compound will exhibit an IC₅₀ with respect to JAK1 and/or JAK3 of about 1 μM to about 50 μM. IC₅₀ is that concentration of inhibitor which reduces the activity of an enzyme (e.g., JAK1 and/or JAK3) to half-maximal level. Certain compounds disclosed herein have been discovered to exhibit inhibition against JAK1 and/or JAK3. In some embodiments, the compounds will exhibit an IC₅₀ with respect to JAK1 and/or JAK3 of no more than about 300 nM. In some embodiments, the compounds will exhibit an IC₅₀ with respect to JAK1 and/or JAK3 of no more than about 1 nM. In certain embodiments, compounds will exhibit an IC₅₀ with respect to JAK1 and/or JAK3 of no more than about 50 μM; in further embodiments, compounds will exhibit an IC₅₀ with respect to JAK1 and/or JAK3 of no more than about 10 μM; in yet further embodiments, compounds will exhibit an IC₅₀ with respect to JAK1 and/or JAK3 of not more than about 5 μM; in yet further embodiments, compounds will exhibit an IC₅₀ with respect to JAK1 and/or JAK3 of not more than about 1 μM, as measured in the JAK1 and/or JAK3 assay described herein.

The phrase “therapeutically effective” is intended to qualify the amount of active ingredients used in the treatment of a disease or disorder or on the effecting of a clinical endpoint.

As used herein, the term “therapeutic” means an agent utilized to treat, combat, ameliorate, prevent or improve an unwanted condition or disease of a patient. In part, embodiments of the present invention are directed to the treatment of JAK1 and/or JAK3-mediated diseases.

The term “therapeutically acceptable” refers to those compounds which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

“Administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with a compound of embodiments herein, can include, but is not limited to, providing the compound onto the target tissue; providing the compound non-systemically to a patient by, e.g., local or topical administration, whereby the therapeutic reaches the target tissue. “Administering” a composition may be accomplished by locally or topically applying the composition to the skin, eye, or other target area.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “therapeutically acceptable salt,” as used herein, represents salts of the compound disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Examples of acids which can be employed to form therapeutically acceptable addition salts include inorganic acids such as hydrochloric, hydrobromic, sulfuric, and phosphoric, and organic acids such as oxalic, maleic, succinic, and citric.

Novel compounds and pharmaceutical compositions, certain of which have been found to inhibit JAK1 and/or JAK3 kinase have been discovered, together with methods of synthesizing and using the compounds including, without limitation, methods for the treatment of JAK1 and/or JAK3 mediated diseases in a patient by topically administering the compounds.

Compounds of the present invention may be selective amongst the JAK1 and/or JAK3 isoforms in various ways. For example, compounds described herein may be selective for JAK1 and/or JAK3 over other isoforms, such as JAK2 and Tyk-2, be a pan-inhibitor of all the isoforms, or be selective for only one isoform. In certain embodiments, compounds of the present invention are selective for JAK1 and/or JAK3 over other isoforms. In some embodiments, the compounds disclosed herein are selective for JAK1 and/or JAK3 over JAK2 and Tyk-2. Selectivity may be determined using enzyme assays, cellular assays or both. In some embodiments, the compounds disclosed herein are at least about 10× selective for JAK1 and/or JAK3 receptors over JAK2 receptor. In some embodiments, the compounds disclosed herein are at least about 10× selective for JAK1 and/or JAK3 receptors over Tyk-2 receptor.

### Preparation of Compound 1

Ethyl (R)-4-((1-(2-cyanoacetyppiperidin-3-yl)amino)-1H-pyrrolo [2,3-b]pyridine-5-carboxylate or “Compound 1” as described herein can be prepared using methods illustrated in synthetic schemes and experimental procedures detailed below. Starting materials used to prepare compounds of the present invention are commercially available or can be prepared using routine methods known in the art. Representative procedures for the preparation of compounds of the invention are outlined in Schemes 1-2 below. Solvents and reagents, whose synthetic preparations are not described below, can be purchased at Sigma-Aldrich or Fisher Scientific.

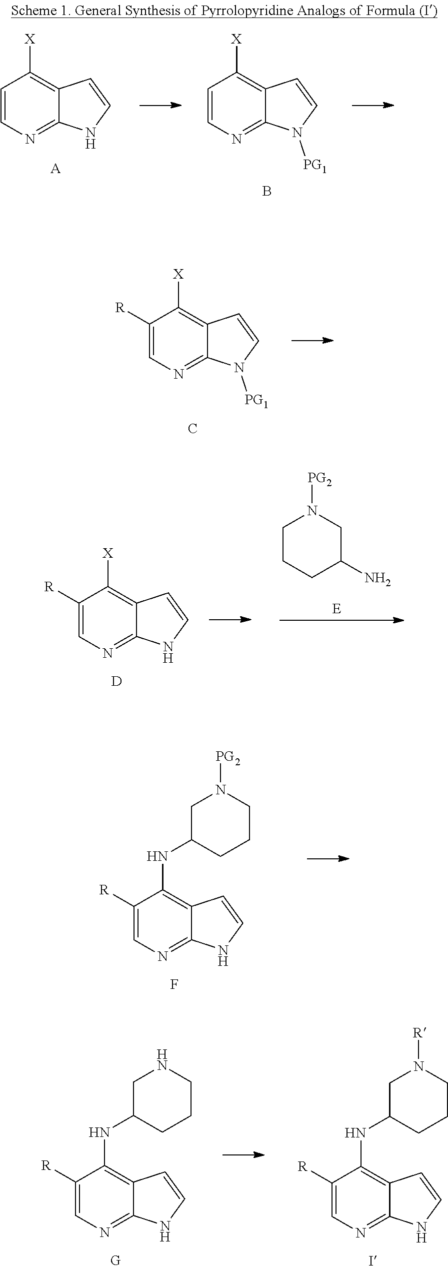

One embodiment of the present application relates to a method of preparing the compound of Formula (I′) having the structure

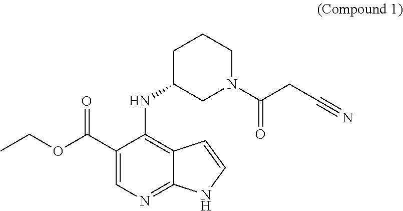 [SMILES: CCOC(=O)c1cnc2[nH]ccc2c1N[C@@H]1CCCN(C(=O)CC#N)C1]

according to the process depicted in Scheme 1 above. This method comprises the steps of:
(a) contacting the compound

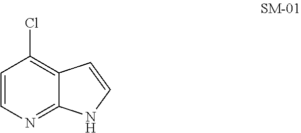

with the compound

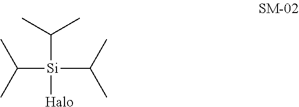

in the presence of a base to form the compound

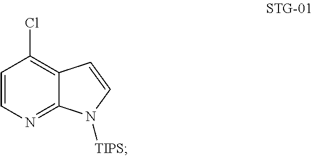

and (b) converting STG-01 to Compound 1.

In some embodiments, the compound SM-02 is triisopropylsilyl chloride.

Any suitable base can be used to carry step (a) above. In one embodiment, the base is NaH.

The step of contacting the compound SM-01 with the compound SM-02 can be conducted in any suitable solvent. Suitable solvents include, without limitation dichloromethane, terahydrofuran (THF), acetonitrile, dimethylformamide (DMF), or dimethyl sulfoxide (DMSO). In one embodiment, the step of contacting the compound SM-01 with the compound SM-02 is conducted in THF.

According to the present invention, the process further comprises the step of contacting the compound STG-01 with ethyl chloro formate in the presence of a base to form the compound

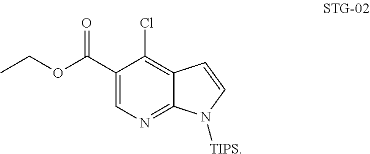

Contacting STG-01 with ethyl chloro formate can be carried out in the presence of any suitable base. For example, suitable bases include, without limitation, methyl lithium, n-butyl lithium, tert-butyl lithium, or sec-butyl lithium. In one embodiment, the base is sec-butyl lithium. In some embodiments, contacting STG-01 with ethyl chloro formate may optionally include diethyl carbonate. In some embodiments, contacting STG-01 with ethyl chloro formate may optionally include N,N,N′,N′-tetramethylethylenediamine (TMEDA).

The step of contacting the compound STG-01 with ethyl chloro formate in the presence of a base can be carried out at a temperature of about −50° C. to about −95° C. for about 30 minutes to about 3 hours. In some embodiments, the contacting is carried out at a temperature of about −60° C. to about −90° C. for about 30 minutes to about 3 hours. In some embodiments, the contacting is carried out at about −60° C. for about 30 minutes to about 3 hours. In some embodiment, the contacting is carried out at a temperature of about −90° C. for about 30 minutes to about 3 hours.

In one embodiment, the step of contacting the compound of STG-01 involves contacting STG-01 with the base at a temperature of −50° C. to about −95° C. for about 30 minutes to about 1.5 hours and then contacting the STG-01-base mixture with ethyl chloroformate at a temperature of −50° C. to about −95° C. for 30 minutes to about 1.5 hours. In one embodiment, the step of contacting the compound of STG-01 involves contacting STG-01 with the sec-butyl lithium at a temperature of −80° C. to about −95° C. for about 30 minutes to about 1.5 hours and then contacting the STG-01-sec-butyl lithium mixture with ethyl chloroformate at a temperature of −80° C. to about −95° C. for 30 minutes to about 1.5 hours.

The step of contacting the compound STG-01 with ethyl chloro format can be conducted in any suitable solvent. Suitable solvents include, without limitation, tetrahydrofuran (THF), dichloromethane, hexane, pentane, benzene, or a mixture thereof. In one embodiment, the reaction is carried out in a solvent selected from the group consisting of THF, hexane, cyclohexane, dimethoxyethane (DME), and mixtures thereof.

According to the present invention, the process can further comprise contacting the compound STG-02 with ethyl alcohol HCl solution to form the compound

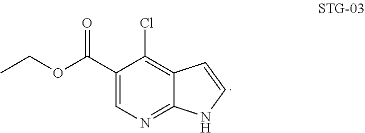

Additionally or alternatively, the process can further comprise contacting the compound STG-02 with an alcoholic HCl solution to form the compound

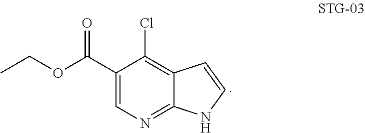

Contacting STG-02 to form STG-03 can be carried out with any suitable alcoholic HCl solutions. Exemplary alcoholic HCl solutions include, without limitation, an ethyl alcohol HCl solution.

According to the present disclosure, the process can further comprise contacting the compound STG-03 with

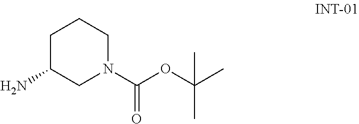

in the presence of an amine base to form the compound

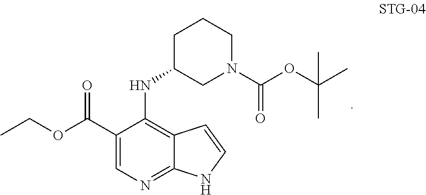 [SMILES: CCOC(=O)c1cnc2[nH]ccc2c1N[C@@H]1CCCN(C(=O)OC(C)(C)C)C1]

Contacting STG-03 with INT-01 can be carried out in the presence of any suitable base. Suitable amine bases include, without limitation diisopropylethylamine (DIPEA), triethylamine, morpholine, piperidine, Na₂CO₃, or KF can be used as a base. In one embodiment, the amine base is diisopropylethylamine (DIPEA). In one embodiment, contacting STG-03 with INT-01 can be carried out in the presence of Pd(OAc)₂, Xantphos, and K₃PO₄.

The step of contacting the compound STG-03 with INT-01 can be conducted in any suitable solvent. Suitable solvents include, without limitation, ethanol, acetonitrile, dimethyl sulfoxide (DMS), dimethylformamide (DMF), water, toluene, xylene, N,N-dimethylacetamide (DMAc), N-methylpyrrolidone (NMP), DME, and mixtures thereof. In one embodiment, the reaction is carried out in ethanol,.

In some embodiments, contacting the compound STG-03 with INT-01 can be conducted in an autoclave at elevated pressure.

In some embodiments, contacting the compound STG-03 with INT-01 can be conducted at a temperature selected from the group consisting of about 80° C., about 90° C., about 100° C., about 110° C., about 120° C., about 130° C., about 140° C., or about 150° C. In one embodiment, the contacting the compound STG-03 with INT-01 is conducted at a temperature about 120° C.

In some embodiments, contacting the compound STG-03 with INT-01 can be conducted for about 10 hours to about 90 hours, about 12 hours to about 24 hours, about 24 hours to about 48 hours, or about 48 hours to about 72 hours. In one embodiment, the contacting is conducted for about 24 hours to about 48 hours.

According to the present invention, the process can further comprise contacting the compound STG-04 with an alcoholic HCl solution to form the compound

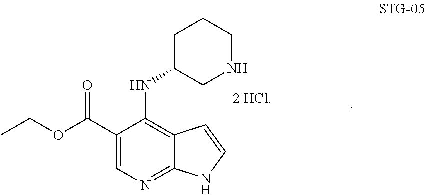 [SMILES: CCOC(=O)c1cnc2[nH]ccc2c1N[C@@H]1CCCNC1]

Contacting STG-04 to form STG-05 can be carried out with any suitable alcoholic HCl solutions. Exemplary alcoholic HCl solutions include, without limitation, an ethyl alcohol HCl solution.

According to the present invention, the process can further comprise contacting the compound STG-05 with

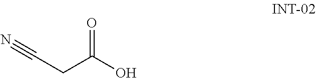

in dichloromethane in the presence of a coupling reagent and an amine base, thereby forming Compound 1.

Contacting STG-05 with INT-02 in dichloromethane can be carried out in the presence of any one or more suitable coupling reagents. Suitable coupling reagents include, without limitation, N-(3-dimethylaminopropyl)-N′-ethylcarbodiimide hydrochloride, N,N′-dicyclohexylcarbodiimide, N,N′-diisopropylcarbodiimide, or N,N′-di-tert-butylcarbodiimide can be used. In one embodiment, the coupling reagent is N-(3-dimethylaminopropyl)-N′-ethylcarbodiimide hydrochloride.

Contacting STG-05 with INT-02 in dichloromethane can be carried out in the presence of any one or more suitable amine bases. Suitable amine bases include, without limitation, diisopropylethylamine (DIPEA), triethylamine, morpholine, or piperidine. In one embodiment, the amine base is diisopropylethylamine (DIPEA).

In some embodiments, contacting STG-05 with INT-02 in dichloromethane is conducted in the presence of hydroxybenzotriazole.

Scheme 2 illustrates a synthesis for Compound 1. Commercially available ethyl 4-chloro-1H-pyrrolo[2,3-b]pyridine-5-carboxylate (CAS #55052-28-3)(SM-01) can be protected using TIPS-Cl under standard reaction conditions using a base such as sodium hydride in a solvent such as DMF or THF followed by the addition of TIPS-Cl. Compound (STG-01) may be selectively lithiated at the 5 position using s-BuLi and reacted with ethyl chloro formate to produce compound (STG-02). Deprotection using an ethyl alcohol HCl solution followed by neutralization can produce the compound (STG-03). Commercially available amine (INT-01) may be used to replace the chloride of compound (STG-03) via nucleophilic aromatic substitution by heating in a solvent such as ethanol in an autoclave at elevated pressure. The resulting Boc protected amine (STG-04) can be deprotected with an ethyl alcohol HCl solution. Once deprotected, amine (STG-05) may be coupled with a carboxylic acid (INT-02) to form Compound 1.

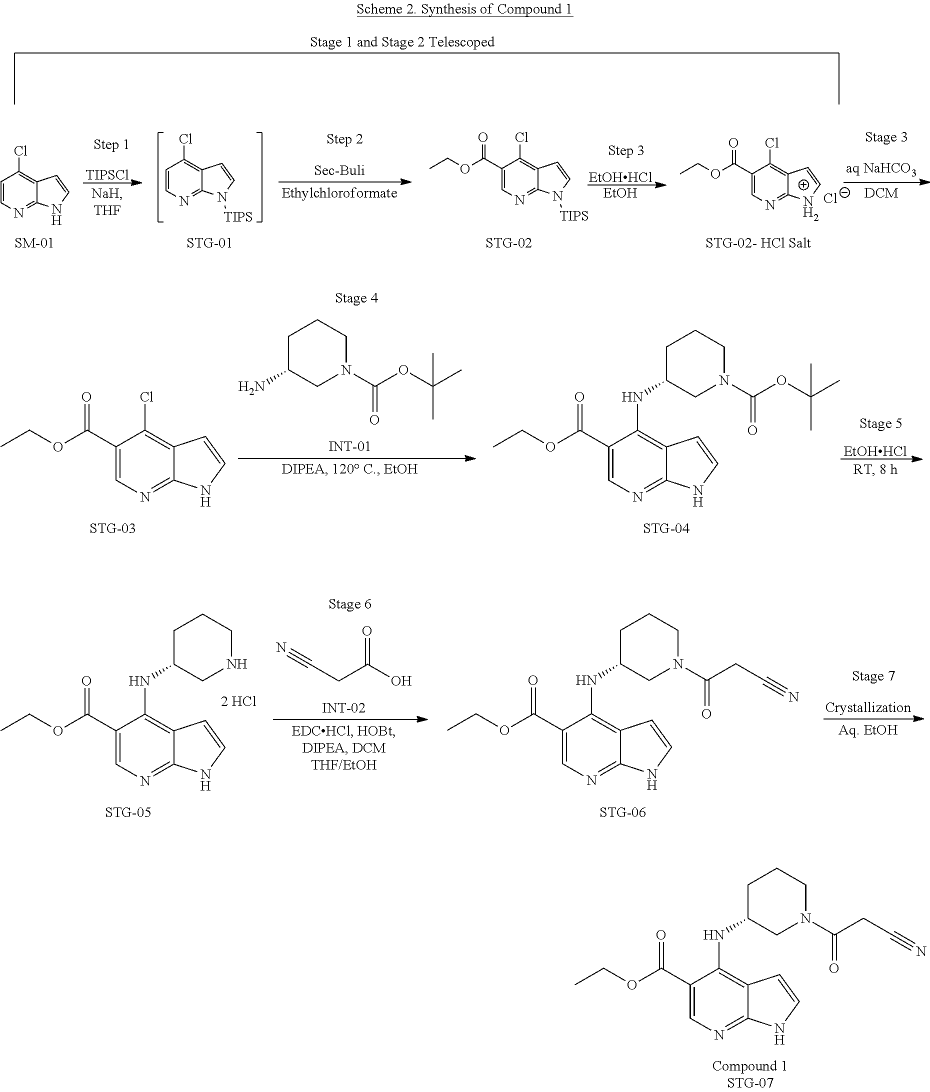

### Preparation of Crystalline Polymorph Form A of Compound 1

Polymorphism is the ability of solid materials to exist in two or more crystalline forms with different arrangements or conformations of the constituents in the crystal lattice. Polymorphism and pseudomorphism are very common amongst drugs and are responsible for differences in many properties. While convention dictates selection of the lowest energy polymorph for incorporation into a formulation due to its chemical stability, considerations must be given to the excipients in the formulation to achieve desired chemical and physical stability and therefore efficacy. Disclosed herein in a particularly useful polymorph of Compound 1, named polymorph Form A, which can be used to prepare or incorporated into topical formulations for treatment of AD, vitiligo, and alopecia areata.

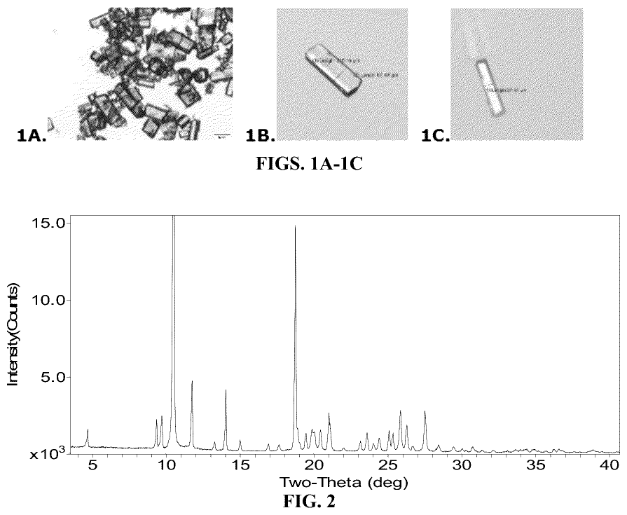

Therefore, in one aspect, the present disclosure provides crystalline polymorph Form A of Compound 1. Crystalline polymorph Form A of Compound 1 is a non-solvated colorless rhombic-dipyramidal crystalline solid. Images of said crystals are shown in FIGS. 1a, 1b, and 1c. Crystalline polymorph Form A of Compound 1 may be characterized as such by powder X-ray diffraction (PXRD) wherein the pattern resulting from the analysis comprises significant peaks at characteristic 2-theta angles. Form A may be characterized, for example, by a significant peak at about 10.50° 2θ. A PXRD pattern of Form A of Compound 1 may further have a significant peak of about 18.86° 2θ. A PXRD pattern of Form A of Compound 1 may further have a significant peak at about one or more of about 9.69° 2θ, about 14.01° 2θ, and about 25.85° 2θ. Yet further, a PXRD pattern of Form A of Compound 1 may additionally have significant peaks at one or more of about 4.67° 2θ, about 9.33° 2θ, about 9.55° 2θ, and about 27.46° 2θ. Parameters that may be used to analyze Compound 1 by PXRD may be found in the Characterization Methods section below.

As stated above, crystalline Form A of Compound 1 is non-solvated crystalline form of Compound 1. In addition to characterization by PXRD, Crystalline Form A of Compound 1 may be also characterized by one or more of thermogravimetric analysis (TGA), differential scanning calorimetry (DSC), and Fourier Transform-Raman (FT-Raman). TGA thermograms, DSC curves, and FT-Raman spectra collected for particular batches of crystalline Form A of Compound 1 are described in the Examples. However, in general, crystalline Form A of Compound 1 may be characterized by a water loss of less than about 1 wt. % when analyzed by TGA. When analyzed via DSC, crystalline Form A of Compound 1 may undergo a phase transition (as evidenced by an endotherm in the DSC) at about 196° C. to about 197° C.

Crystalline polymorph Form A of Compound 1 may be characterized by a FT-Raman spectra comprising a significant peak at about 1499.7 cm⁻¹. A FT-Raman spectra of Form A of Compound 1 may further have a significant peak at about 31.867 cm⁻¹. A FT-Raman spectra of Form A of Compound 1 may further have a significant peak at one or more of about 28.008 cm⁻¹, about 27.729 cm⁻¹, about 20.742 cm⁻¹, and about 19.862 cm⁻¹. A FT-Raman spectra of Form A of Compound 1 may further have a significant peak at one or more of about 17.799 cm⁻¹, about 17.727 cm⁻¹, about 17.47 cm⁻¹, and about 16.713 cm⁻¹.

Crystalline polymorph Form A of Compound 1 may be characterized by a PXRD pattern comprising a significant peak at a 2θ angle of about 10.50° and characterized by a FT-Raman spectra comprising a significant peak at about 1499.7 cm⁻¹. Crystalline polymorph Form A of Compound 1 may be further characterized by a PXRD comprising a significant peak at 2θ angle of about 18.86° and a FT-Raman spectra further comprising a significant peak at about 31.867 cm⁻¹. Crystalline polymorph Form A of Compound 1 may be further characterized by a PXRD comprising a significant peak at a 2θ angle at one or more of about 9.69°, about 14.01°, and about 25.85° and a FT-Raman spectra further comprising a significant peak at one or more of about 28.008 cm⁻¹, about 27.729 cm⁻¹, about 20.742 cm⁻¹, and about 19.862 cm⁻¹. Crystalline polymorph Form A of Compound 1 may be further characterized by a PXRD comprising a significant peak at a 2θ angle at one or more of about 9.33°, about 9.55°, and about 27.46° and a FT-Raman spectra further comprising a significant peak at one or more of about 17.799 cm⁻¹, about 17.727 cm⁻¹, about 17.47 cm⁻¹, and about 16.713 cm⁻¹.

Crystalline Form A of Compound 1 may be prepared by mixing ethyl (R)-4-((1-(2-cyanoacetyl)piperidin-3-yl)amino)-1H-pyrrolo[2,3-b]pyridine-5 -carboxylate in a lower alcohol, (e.g., ethanol) under an inert atmosphere (e.g., nitrogen). The starting ethyl (R)-4-((1-(2-cyanoacetyl)piperidin-3-yl)amino)-1H-pyrrolo[2,3-b]pyridine-5-carboxylate material may be any form (e.g., crystalline, amorphous, solvated) to form a reaction mixture. The reaction mixture may be heated and stirred to obtain a clear solution. Heating may be carried out until dissolution is achieved, for example, 2 hours to 3 hours. The reaction mixture may be heated to facilitate dissolution, for example, to a temperature of about 70° C. to about 75° C. Optionally, activated carbon may be added to the heated reaction mixture while maintaining an elevated temperature of about 70° C. to about 75° C. The reaction mixture may continue to be stirred, for example, for about 1 hour to about 2 hours.

Optionally, after 1 hours to 2 hours, the reaction mixture may be filtered at 70° C. to about 75° C. to remove the activated carbon. Filtration may be carried out by any method known in the art. The reaction mixture may be further passed through a 0.2-micron filter while maintaining the elevated temperature of about 70° C. to about 75° C. Stirring may continue for up to about 1 hours to about 2 hours while maintaining the reaction mixture at 70° C. to about 75° C.

The reaction mixture may then be cooled to a temperature of about 10° C. to about 15° C. to yield Compound 1 in its polymorph Form A crystalline form. Optionally, seed crystals of crystalline polymorph Form A of Compound 1 may be added to facilitate the development of the target polymorph in the reaction mixture.

The cooling may be carried out in a step-wise fashion over a period of time of up to 2 days (48 hours). For example, the clear solution at 70° C. to about 75° C., which has optionally been treated with activated carbon and filtered, may, after about 1 hour to about 2 hours of mixing, be slowly cooled to a temperature of about 40° C. to about 45° C. The cooling may take place over a period of time, for example, of about 3 hours to 4 hours. The reaction mixture may be maintained at this temperature for a prolonged period, for example, about 6 hours to about 7 hours.

In a second cooling step, the reaction mixture may be further slowly cooled to about 25° C. to about 30° C., for example, over a period of about 3 hours to about 4 hours. The reaction mixture may be maintained at this temperature for a prolonged period, for example, about 12 hours to about 14 hours, during which the reaction mixture may be stirred.

In a third cooling step, the reaction mixture may then be further slowly cooled to about 10° C. to about 15° C., for example, over a period of about 2 hours to about 3 hours. The reaction mixture may be maintained at this temperature for a prolonged period, for example, for about 4 hours to about 6 hours, during which the reaction mixture may be stirred.

Crystalline polymorph Form A of Compound 1 may then be isolated, for example, by filtering the reaction mixture to isolate a solid which has formed. The solid may be dried, for example, under vacuum at about 50° C. to about 55° C. for about 22 hours to about 24 hours to yield Compound 1 in its polymorph Form A crystalline form.

### Characterization Methods

X-Ray Data Collection: A single, rod-like crystal (0.050×0.0932×0.38 mm), as shown in FIG. 1a, 1b, and 1c, was mounted on a MiTeGen™ cryo-loop. Preliminary analysis and data collection were performed at temperature of 200 K using copper Kα radiation (1.54184 Å) with a Bruker APEX II Duo™ diffractometer equipped with a IμS Cu source and an Oxford Crystalstream™ low temperature device.

X-Ray Structure Determination: Data form X-Ray Data Collection (above) was integrated using an orthorhombic unit cell. The structure was determined and refined using the Bruker SHELXTL software package, using the space group P 2₁ 2₁ 2₁, with Z=4 for the formula unit, C₁₈H₂₁N₅O₃.

Simulated PXRD: The program Mercury 4.0 was used to analyze the structural coordinates. The command “calculate powder pattern” was used to create a representative PXRD pattern. The h, k, l, and 2θ values were identified using the software and compared with experimental data. Values for d-spacing were generated using Apex3 v. 2019.1 software.

Powder X-Ray Diffraction: A ˜50 mg sample of Compound 1, Form A was ground to a uniform, find powder in an agate mortar and pestle and carefully packed into a sample holder. The PXRD measurements were conducted at room temperature using a PANalytical X'Pert Pro MPD diffractometer. PXRD measurements were conducted using Ni-filtered copper CuKα radiation with a wavelength of 1.54 Å over an angle range of 2θ=4°−42° and a step size of 0.02°.

Differential Scanning calorimetry (DSC): DSC was conducted with a TA instruments Q100 or Q2000 differential scanning calorimeter equipped with an autosampler and a refrigerated cooling system under 30 mL/min N₂ purge. DSC thermograms of samples were obtained at 10° C./min in crimped Al pans.

Thermogravimetric Analysis: TGA thermograms were obtained with a TA Instruments Q50 thermogravimetric analyzer under 40 mL/min N₂ purge in Pt or Al pans. TGA thermograms of samples were obtained at 10° C./min in crimped Al pans. TGA analysis with IR Off-Gas Detection (TGA-IR) was conducted with a TA Instruments Q5000 thermogravimetric analyzer interfaced to a Nicolet 6700 FT-IR spectrometer equipped with an external TGA-IR module with a gas flow cell and DTGS detector. TGA was conducted under 60 mL/min N₂ flow and heating rate of 15° C./min in Pt or Al pans. IR spectra were collected at 4 cm⁻¹ resolution and 32 scans at each time point.

FT-Raman Spectroscopy: Raman spectra were collected with a Nicolet NXR9650 or NXR 960 spectrometer equipped with 1064 nm Nd:YVO₄ excitation laser, InGaAs and liquid-N₂ cooled Ge detectors, and a microstage. All spectra were acquired at 4 cm⁻¹ resolution, 64-128 scans, using Happ-Genzel application function and 2-level zero-filling.

### Compounds

Embodiments herein are directed to a crystalline form of ethyl (R)-4-((1-(2-cyanoacetyl)piperidin-3-yl)amino)-1H-pyrrolo [2,3 -b]pyridine-5 -carboxylate (Compound 1), which has been found to inhibit JAK1 and/or JAK3 Kinase, together with methods of synthesizing and using this crystalline form. Some embodiments include methods for the treatment of diseases in a patient by topically administering the crystalline form of Compound 1 as described herein or a pharmaceutical composition prepared from the crystalline polymorph Form A of Compound 1.

The crystalline form of Compound 1 or a pharmaceutical composition prepared from the crystalline polymorph Form A of Compound 1 disclosed herein possesses useful JAK1 and/or JAK3 inhibiting activity and may be used in the treatment or prophylaxis of a disease or condition in which JAK1 and/or JAK3 plays an active role. Thus, embodiments are also directed to pharmaceutical compositions comprising the crystalline form disclosed herein or prepared from the crystalline form disclosed herein together with a pharmaceutically acceptable carrier, as well as methods of making and using the crystalline form and compositions comprising the same. Certain embodiments are directed to methods for inhibiting JAK1 and/or JAK3. Other embodiments are directed to methods for treating a JAK1 and/or JAK3-mediated disorder in a patient in need of such treatment, comprising administering to said patient a therapeutically effective amount of the crystalline form or composition prepared from the crystalline form disclosed herein according to the present invention. Also provided is the use of the crystalline form disclosed herein in the manufacture of a medicament for the treatment of a disease or condition ameliorated by the inhibition of JAK1 and/or JAK3.

Also provided are embodiments wherein any embodiment herein may be combined with any one or more of the other embodiments, unless otherwise stated and provided the combination is not mutually exclusive.

The crystalline form of embodiments herein may also refer to a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination of the foregoing of the compounds of embodiments herein.

The crystalline form of Compound 1 described herein may contain a stereogenic center and may be chiral and thus exist as enantiomers. Where the compounds according to embodiments herein possess two or more stereogenic centers, they may additionally exist as diastereomers. Embodiments herein includes all possible stereoisomers as substantially pure resolved enantiomers, racemic mixtures thereof, as well as mixtures of diastereomers. In some embodiments, the formulas are shown without a definitive stereochemistry at certain positions. Embodiments herein includes all stereoisomers of such formulas and pharmaceutically acceptable salts thereof. Diastereomers may be separated by, for example, fractional crystallization from a suitable solvent, and pairs of enantiomers thus obtained may be separated into individual stereoisomers by conventional means, for example by the use of an optically active acid or base as a resolving agent or on a chiral HPLC column. Further, any enantiomer or diastereomer of the crystalline form may be obtained by stereospecific or stereoselective synthesis using optically pure or enantioenriched starting materials or reagents of known configuration. The scope of embodiments herein as described and claimed encompasses the racemic forms of the compounds as well as the individual enantiomers, diastereomers, and stereoisomer-enriched mixtures.

Suitable pharmaceutically acceptable acid addition salts of the crystalline form herein may be prepared from an inorganic acid or an organic acid. All of these salts may be prepared by conventional means from the corresponding compound of embodiments herein by treating, for example, the compound with the appropriate acid or base.

Pharmaceutically acceptable acids include both inorganic acids, for example hydrochloric, hydrobromic, hydroiodic, nitric, carbonic, sulfuric, phosphoric and diphosphoric acid; and organic acids, for example formic, acetic, trifluoroacetic, propionic, succinic, glycolic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, galacturonic, citric, fumaric, gluconic, glutamic, lactic, maleic, malic, mandelic, mucic, ascorbic, oxalic, pantothenic, succinic, tartaric, benzoic, acetic, xinafoic (1-hydroxy-2-naphthoic acid), napadisilic (1,5-naphthalenedisulfonic acid) and the like.

The crystalline form herein may exist in both unsolvated and solvated forms. The term solvate is used herein to describe a molecular complex comprising the crystalline form herein and an amount of one or more pharmaceutically acceptable solvent molecules. The term hydrate is employed when said solvent is water. Examples of solvate forms include, but are not limited to, the crystalline form as disclosed herein in association with water, acetone, dichloromethane, 2-propanol, ethanol, methanol, dimethylsulfoxide (DMSO), ethyl acetate, acetic acid, ethanolamine, or mixtures thereof. It is specifically contemplated that in embodiments herein one solvent molecule can be associated with one molecule of the crystalline form disclosed herein, such as a hydrate.

Furthermore, it is specifically contemplated that in embodiments herein, more than one solvent molecule may be associated with one molecule of the crystalline form, such as a dihydrate. Additionally, it is specifically contemplated that in embodiments herein less than one solvent molecule may be associated with one molecule of the crystalline form of embodiments herein, such as a hemihydrate. Furthermore, solvates of embodiments herein are contemplated as solvates of compounds of embodiments herein that retain the biological effectiveness of the non-solvate form of the compounds.

The compounds disclosed herein can exist as and therefore include all stereoisomers, conformational isomers and mixtures thereof in all proportions as well as isotopic forms such as deuterated compounds.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

### Pharmaceutical Compositions

Some embodiments herein are directed to a pharmaceutical composition comprising the crystalline form of Compound 1 or a pharmaceutical composition prepared from the crystalline polymorph Form A of Compound 1 and a pharmaceutically acceptable carrier or diluent.

Also provided is a pharmaceutical composition comprising the crystalline form of Compound 1 as disclosed herein or a pharmaceutical composition prepared from the crystalline polymorph Form A of Compound 1 as disclosed herein in a non-aqueous formulation, together with a pharmaceutically acceptable carrier.

In some embodiments, the pharmaceutical compositions for use in accordance with embodiments herein can be formulated in conventional manner using one or more physiologically acceptable carriers or excipients.

The formulations include those suitable for topical (including, for example, dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association the crystalline form disclosed herein (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

The crystalline form disclosed herein or a pharmaceutical composition prepared from the crystalline form disclosed herein may be administered topically, that is by non-systemic administration. This includes the application of the crystalline form disclosed herein or a pharmaceutical composition prepared from the crystalline form disclosed herein externally to the epidermis or the buccal cavity and the instillation of such a crystalline form into the ear, eye and nose.

In some embodiments, the crystalline form disclosed herein or a pharmaceutical composition prepared from the crystalline form disclosed herein may be administered ophthalmically. In some embodiments, the crystalline form disclosed herein or a pharmaceutical composition prepared from the crystalline form disclosed herein may be administered as an ophthalmic composition. The crystalline form of embodiments herein or a pharmaceutical composition prepared from the crystalline form disclosed herein may be administered as, for example, liquid preparations, including eye lotions, spray, or eye drops for topical administration. In some embodiments, the crystalline form disclosed herein or a pharmaceutical composition prepared from the crystalline form disclosed herein may be administered as semi-solid preparations, for example, applied to the eyelid, such as cream, lotion, gel, ointment, or paste. In some embodiments, the crystalline form disclosed herein or a pharmaceutical composition prepared from the crystalline form disclosed herein may be administered as solid dosage forms, for example, applied to the eye surface to produce modified release, such as a powder. In some embodiments, the crystalline form of embodiments herein or a pharmaceutical composition prepared from the crystalline form disclosed herein is administered through devices for surgical implantation, parenteral products, (e.g., intracorneal or intravitreous products), liquids for irrigation, or the like. In some embodiments, the composition comprising the crystalline form disclosed herein or prepared from the crystalline form disclosed herein are sterile and free from particulate matters. In some embodiments, the crystalline form disclosed herein or a pharmaceutical composition prepared from the crystalline form disclosed herein may be administered by intraocular injection, intraorbital injection, or an intravitreal injection. In some embodiments, the intraocular injection may be to the anterior chamber of the eye, posterior chamber of the eye, or a combination thereof. For example, the crystalline form disclosed herein or a pharmaceutical composition prepared from the crystalline form disclosed herein may be administered to the posterior intraorbital region of the eye.

In some embodiments, formulations suitable for topical administration include liquid or semi-liquid preparations suitable for penetration into the skin to the site of inflammation such as a solution, powder, fluid emulsion, fluid suspension, semi-solid, ointment, paste, cream, gel, jelly, foam, liniment, lotion, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

In some embodiments, the formulation suitable for topical administration is a non-aqueous solution, suspension, or emulsion.

Gels for topical or transdermal administration may comprise, generally, a mixture of volatile solvents, nonvolatile solvents, and water. In certain embodiments, the volatile solvent component of the buffered solvent system may include lower (C₁-C₆) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question.

The crystalline form or a pharmaceutical composition prepared from the crystalline form disclosed herein may be administered at a dose of from 0.1 to 500 mg/kg per day. The dose range for adult humans is generally from 5 mg to 2 g/day.

The amount of active ingredient that may be combined with the carrier materials to produce a single dosage form will vary depending upon the host treated and the particular mode of administration.

When employed as pharmaceuticals, the crystalline form can be administered in the form of pharmaceutical compositions. These compositions can be prepared in a manner well known in the pharmaceutical arts, and can be administered by a variety of routes, depending upon whether local treatment is desired and upon the area to be treated. Administration of the disclosed crystalline form or compositions prepared from the crystalline form disclosed herein may be topical (including dermal, buccal, sublingual and intraocular). Pharmaceutical compositions for topical administration may include foams, transdermal patches, ointments, lotions, creams, gels, solutions, fluid emulsions, fluid suspensions, semi-solids, pastes, drops, suppositories, sprays, liquids and powders. Conventional pharmaceutical carriers, aqueous, powder or oily bases, thickeners and the like may be necessary or desirable. Coated condoms, gloves and the like may also be useful. In some embodiments, the crystalline forms can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The artisan can refer to various pharmacologic references for guidance. For example, Modern Pharmaceutics, 5th Edition, Banker & Rhodes, CRC Press (2009); and Goodman & Gilman's The Pharmaceutical Basis of Therapeutics, 13th Edition, McGraw Hill, New York (2018) can be consulted.

In some embodiments, the pharmaceutical composition is a solution.

In some embodiments, a method of treating a JAK1 and/or JAK3 mediated disease administering a pharmaceutical composition of embodiments disclosed herein. In some embodiments, the crystalline form is in a therapeutically effective amount. In some embodiments, the therapeutically effective amount is an amount disclosed herein.

Some embodiments disclosed herein also include pharmaceutical compositions which contain, as the active ingredient, the crystalline form disclosed herein in combination with one or more pharmaceutically acceptable carriers (excipients).

In some embodiments, a method of making a pharmaceutical composition comprises mixing the active ingredient with an excipient, diluting the active ingredient using an excipient, or enclosing the active ingredient within a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of powders, elixirs, suspensions, emulsions, solutions, syrups, aerosols (as a solid or in a liquid medium), ointments containing, for example, up to 10% by weight of the crystalline form, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose, including eutectic solvents, eutectic-based ionic liquids, or ionic liquids. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions can be formulated so as to provide quick, sustained or delayed release of the active ingredient after administration to the patient by employing procedures known in the art.

The compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The crystalline form or a pharmaceutical composition prepared from the crystalline form can be effective over a wide dosage range and can be generally administered in a therapeutically effective amount. It will be understood, however, that the amount of the crystalline form or a pharmaceutical composition prepared from the crystalline form actually administered will usually be determined by a physician, according to the relevant circumstances, including the condition to be treated, the chosen route of administration, the actual crystalline form or a pharmaceutical composition prepared from the crystalline form administered, the age, weight, and response of the individual patient, the severity of the patient's symptoms, and the like.

In some embodiments, the pharmaceutical composition may comprise about 0.01% to about 50% of the crystalline form disclosed herein. In some embodiments, the crystalline form is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the composition. In some embodiments, the composition is suitable for topical administration. In some embodiments, the composition is suitable for oral, parenteral (including subcutaneous, intradermal, intramuscular, intravenous, intraarticular, and intramedullary), intraperitoneal, intrathecal, intradural, transmucosal, transdermal, rectal, intranasal, topical (including, for example, dermal, buccal, sublingual and intraocular), intravitreal, or intravaginal administration.

In some embodiments, the crystalline form is in a therapeutically effective amount. In some embodiments, the therapeutically effective amount may be about 1 mg to about 1000 mg, about 1 mg to about 900 mg, about 1 mg to about 800 mg, about 1 mg to about 700 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 10 mg to about 1000 mg, about 50 mg to about 1000 mg, about 100 mg to about 1000 mg, about 200 mg to about 1000 mg, about 300 mg to about 1000 mg, about 400 mg to about 1000 mg, about 500 mg to about 1000 mg, about 10 mg to about 500 mg, about 50 mg to about 500 mg, about 100 mg to about 500 mg, about 10 mg to about 300 mg, about 50 mg to about 300 mg, from about 100 mg to about 300 mg, about 10 mg to about 150 mg, about 50 mg to about 150 mg, about 60 mg to about 120 mg, about 50 mg to about 120 mg or a range between any two of these values. Specific examples include, for example, about 1000 mg, about 900 mg, about 800 mg, about 700 mg, about 750 mg, about 600 mg, about 500 mg, about 400 mg, about 450 mg, about 300 mg, about 250 mg, about 200 mg, about 175 mg, about 150 mg, about 125 mg, about 120 mg, about 110 mg, about 100 mg, about 90 mg, about 80 mg, about 70 mg, about 60 mg, about 50 mg, about 30 mg, about 20 mg, or any value between the ranges disclosed above.

In some embodiments, the therapeutically effective amount can vary according to, for example, the particular use for which the treatment is made, the manner of administration of the crystalline form or a pharmaceutical composition prepared from the crystalline form, the health and condition of the patient, and the judgment of the prescribing physician. The proportion or concentration of a crystalline form in a pharmaceutical composition can vary depending upon a number of factors including dosage, chemical characteristics (e.g., hydrophobicity), and the route of administration. For example, the crystalline form can be provided in an aqueous physiological buffer solution containing about 0.1 to about 10% w/v of the crystalline form for parenteral administration. Some typical dose ranges for the crystalline forms are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the crystalline form selected, formulation of the excipient, and its route of administration. Effective doses can be extrapolated from dose-response curves derived from in vitro or animal model test systems.

The amount of crystalline form or composition prepared from the crystalline form administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications.

For preparing solid compositions, the principal active ingredient can be mixed with a pharmaceutical excipient to form a solid pre-formulation composition containing a homogeneous mixture of the crystalline form disclosed herein. When referring to these pre-formulation compositions as homogeneous, the active ingredient is typically dispersed evenly throughout the composition so that the composition can be readily subdivided into equally therapeutically effective unit dosage forms. This solid pre-formulation is then subdivided into unit dosage forms of the type described above containing from, for example, about 0.1 to about 1000 mg of the active ingredient.

In some embodiments, the compositions administered to a patient can be in the form of pharmaceutical compositions described above. In some embodiments, these compositions can be sterilized by conventional sterilization techniques, or may be sterile filtered. Aqueous solutions can be packaged for use as is, or lyophilized, the lyophilized preparation being combined with a sterile aqueous carrier prior to administration. In some embodiments, the pH of the crystalline form preparations is about 3 to about 11, about 5 to about 9, about 5.5 to about 6.5, or about 5.5 to about 7.5. It will be understood that use of certain of the foregoing excipients, carriers, or stabilizers will result in the formation of pharmaceutical salts.

### Methods of Use

The present invention relates to a method of modulating a JAK1 and/or JAK3-mediated function in a subject comprising the topical or local administration of a therapeutically effective amount of the crystalline form of Compound 1 disclosed herein or a pharmaceutical composition prepared from the crystalline form disclosed herein.

Also provided herein is a method of treating a JAK1 and/or JAK3-mediated skin or ocular condition, where the method comprises locally administering to a patient in need thereof a therapeutically effective amount of the crystalline form as disclosed herein or a pharmaceutical composition prepared from the crystalline form disclosed herein. In certain embodiments, the therapeutically effective amount of the crystalline form as disclosed herein may be in the form of a pharmaceutical composition. In embodiments, the pharmaceutical composition may include a pharmaceutically acceptable excipient.

Diseases or disorders associated with a JAK1 kinase and/or a JAK3 kinase that are treated by crystalline forms or a pharmaceutical composition prepared from the crystalline form of the present invention include autoimmune skin and ocular conditions, chronic inflammatory skin and ocular conditions, acute inflammatory skin and ocular conditions, and auto-inflammatory skin and ocular conditions. Thus, in some embodiments, the present invention provides a method for treating a JAK1 and/or JAK3 mediated skin or ocular condition in a patient in need thereof, wherein said method comprises topically or ocularly administering to said patient a therapeutically effective amount of a provided crystalline form or a composition prepared from the crystalline form. Such JAK1 and/or JAK3-mediated diseases or disorders include, but are not limited to, those described herein.

In some embodiments, said JAK1 and/or JAK3-mediated disease or disorder is a skin disorder or dermatoses selected from pruritus, a hair loss disorder, a chronic or acute inflammatory skin condition, an autoimmune skin condition, an infectious skin condition (e.g., bacterial or viral skin infection), dermatitis (e.g., atopic dermatitis/eczema, allergic dermatitis, contact dermatitis, photodermatitis, seborrheic dermatitis, stasis dermatitis, acute febrile neutrophilic dermatosis (Sweet's syndrome), chronic atypical neutrophilic dermatosis with lipodystrophy and elevated temperature syndrome (CANDLE Syndrome), psoriasis, acne, skin sensitization, skin irritation, skin rash, skin allergy, allergic contact sensitization, vitiligo (e.g., segmental vitiligo, non-segmental vitiligo, centrofacial vitiligo, mucosal vitiligo, confetti vitiligo, trichrome vitiligo, marginal inflammatory vitiligo, quadrichrome vitiligo, blue vitiligo, Koebner phenomenon, vulgaris vitiligo, generalized vitiligo, universal vitiligo, mixed vitiligo, focal vitiligo, solitary mucosal vitiligo), and alopecia (e.g., alopecia areata, patchy alopecia areata, alopecia totalis, alopecia universalis, ophiasis pattern alopecia areata, sisaihpo pattern alopecia areata, androgenetic alopecia, telogen effluvium, tinea capitis, hypotrichosis, hereditary hypotrichosis simplex, scarring alopecia, lichen planopilaris, central centrifugal cicatricial alopecia, or frontal fibrosing alopecia).

Also provided herein is the crystalline form of Compound 1 as disclosed herein for use as a medicament.

Also provided herein is the crystalline form of Compound 1 as disclosed herein for use as a medicament for the treatment of a JAK1 and/or JAK3-mediated skin or ocular condition.

Also provided is the use of the crystalline form of Compound 1 as disclosed herein as a medicament.

Also provided is the use of the crystalline form of Compound 1 as disclosed herein as a medicament for the treatment of a JAK1 and/or JAK3-mediated skin or ocular disease.

Also provided is the crystalline form Compound 1 as disclosed herein for use in the manufacture of a medicament for the treatment of a JAK1 and/or JAK3-mediated skin or ocular condition.

Also provided is the use of the crystalline form Compound 1 as disclosed herein for the treatment of a JAK1 and/or JAK3-mediated skin or ocular condition.

Also provided herein is a method of inhibition of JAK1 and/or JAK3 comprising administering the crystalline form as disclosed herein. In any embodiment, said administering is carried out locally, topically, or ocularly.

Thus, in another aspect, certain embodiments provide methods for treating JAK1 and/or JAK3-mediated disorders in a human or animal subject in need of such treatment comprising administering to said subject an amount of a crystalline form disclosed herein effective to reduce or prevent said disorder in the subject, in combination with at least one additional agent for the treatment of said disorder that is known in the art. In a related aspect, certain embodiments provide therapeutic compositions comprising at least one crystalline form disclosed herein in combination with one or more additional agents for the treatment of JAK1 and/or JAK3-mediated disorders.

In certain embodiments, the topically administered JAK1 and/or JAK3 inhibitor/antagonist described herein can be used for the treatment of alopecia areata (e.g. patchy alopecia areata, alopecia totalis, alopecia universalis) alone or in combination with topical or intralesional corticosteroids, topical minoxidil, oral finasteride, oral dutasteride, contact sensitization therapy such as with squaric acid dibutyl ester, dinitrochlorobenzene, diphencyprone, topical or oral methoxalen and ultraviolet a (PUVA), topical anthralin, hair transplantation procedures, or other therapies known to have beneficial effects in the condition.

In certain embodiments, the topically administered JAK1 and/or JAK3 inhibitor/antagonist disclosed herein can be used for the treatment of male or female-pattern baldness (androgenetic alopecia) alone or in combination with topical minoxidil, oral finasteride (in male), oral dutasteride (in male), topical antiandrogens, hair transplantation procedures, or other therapies known to have beneficial effects in the condition.

In certain embodiments, the crystalline forms may be used for the treatment of vitiligo (e.g. localized vitiligo, focal vitiligo, generalized vitiligo, segmental vitiligo, acral vitiligo, facial vitiligo, acrofacial vitiligo, mucosal vitiligo, confetti vitiligo, trichrome vitiligo, marginal inflammatory vitiligo, quadrichrome vitiligo, blue vitiligo, Koebner phenomenon, vulgaris vitiligo, mixed acrofacial and vulgaris vitiligo, or universal vitiligo) alone or in combination with topical corticosteroids, topical tacrolimus, topical pimecrolimus, phototherapy such as ultraviolet light therapy with UVB, narrow-band UVB, oral or topical psoralen plus ultraviolet A (PUVA), calcipotriene or other topical vitamin D analogs, excimer laser phototherapy, systemic immunosuppressive agents, surgical treatments such as skin minigrafting, transplantation of autologous epidermal suspension, camouflage such as with make-up or dihydroxyacetone and such, or other therapies known to have beneficial effects in the condition.

Besides being useful for human treatment, certain crystalline forms and formulations disclosed herein may also be useful for veterinary treatment of companion animals, exotic animals and farm animals, including mammals, rodents, and the like. More preferred animals include horses, dogs, and cats.

To facilitate a better understanding of the embodiments of the present invention, the following examples of preferred or representative embodiments are given. In no way should the following examples be read to limit, or to define, the scope of the invention.

### EXAMPLES

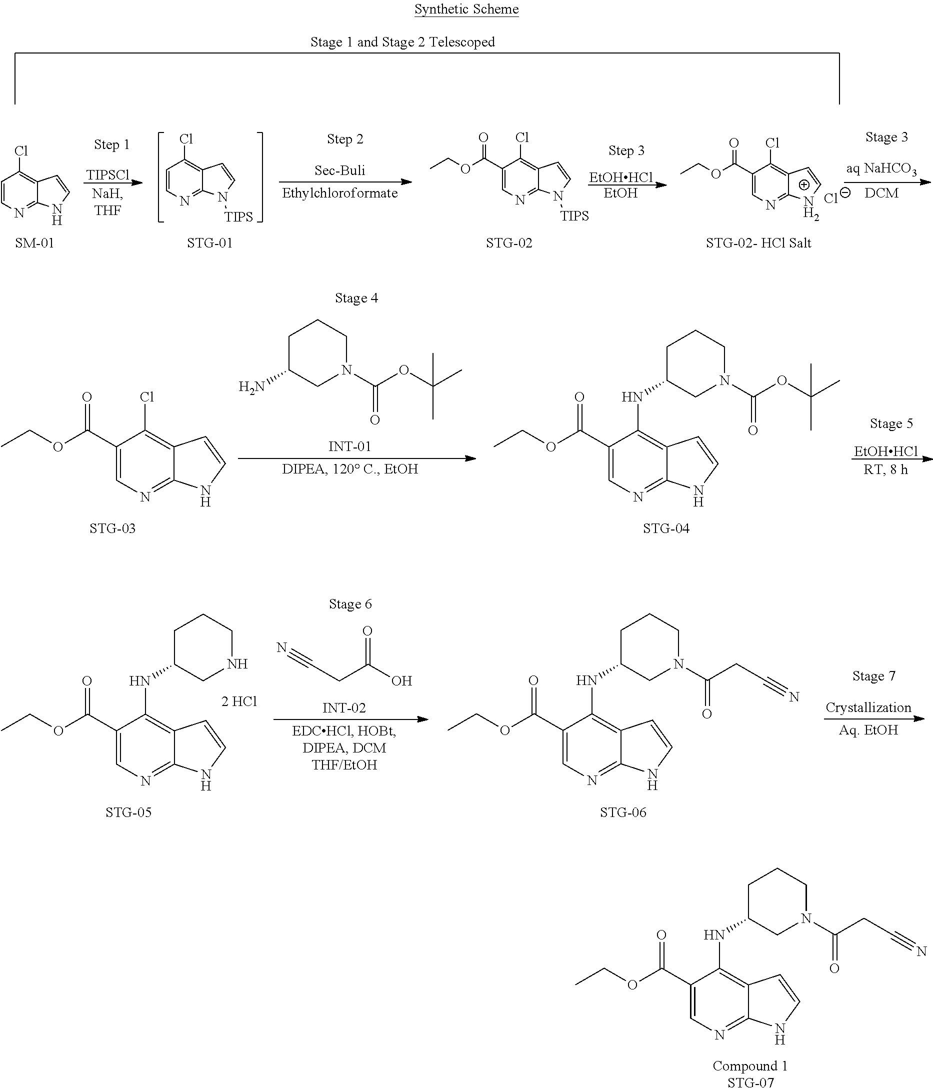

### Example 1: Preparation of ethyl 4-chloro-1H-pyrrolo[2,3-b]pyridine-5-carboxylate hydrochloride (STG-02 HCl Salt)

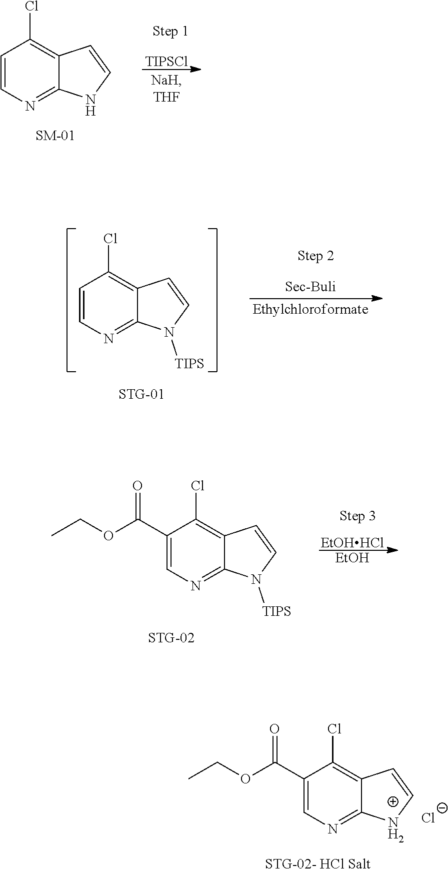

To a suspension of NaH (2.01 Kg, 60% oil dispersion, 1.4 eq.) in THF (55.0 L, 10.0 vol.) was added 4-chloro-1H-pyrrolo[2,3-b] pyridine (SM-01, 5.5 Kg, 1.0 eq.) in THF (110.0 L, 20.0 vol.) slowly at 25±5° C. Reaction mass was cooled to 5±5° C. and Tri-isopropylsilyl chloride (8.33 kg, 1.2 eq.) was added at same temperature. The reaction mass temperature was raised to 25±5° C. and stirred for 1-2 h, progress of the reaction was monitored by TLC. Reaction mass was cooled to −87.5±7.5°, slowly added Sec-BuLi (23.79 Kg, 1.4M in cyclohexane solution, 1.2 eq.) at −87.5±7.5° C. under nitrogen atmosphere. Stirred the reaction mass at −87.5±7.5° C. for 1 h±15 min under nitrogen atmosphere. A mixture of Ethyl Chlorofomate (5.88 Kg, 1.5 eq.) in THF (5.5 L, 1.0 vol) was added slowly into the reaction mass at −87.5±7.5° C. (addition is exothermic) under nitrogen atmosphere. The reaction mixture was stirred for 1 h±15 min at −87.5±7.5° C. under nitrogen atmosphere. Reaction was monitored by HPLC (Limit NMT 5.0% by HPLC). After completion of reaction, aq. Ammonium chloride solution (8.0 vol.) was slowly added at −87.5 to −40° C. under nitrogen atmosphere. Reaction mass temperature was increased to 25±5° C. and it was extracted with MTBE (2×6.0 vol.) at 30±5° C. The organic layer was washed with purified water (5.0 vol.) at 30±5° C. followed by washing with aqueous sodium chloride solution (10%, 5.0 vol.). Organic layer was evaporated under reduced pressure to obtain the crude (16.5 Kg). Ethanol (2.0 vol.) was added to the crude compound at 25±5° C. EtOH·HCl (27.5 L, 5M, 20%, 5.0 vol.) was added slowly at 25±5° C. and stirred for 1-2 h. The resulting solids were filtered, washed with ethanol (1-2 vol.) and dried under vacuum at 45±5° C. to obtain the ethyl 4-chloro-1H-pyrrolo[2,3-b]pyridine-5-carboxylate hydrochloride (STG-01) as an off-white solid (7.25 Kg, 77%). ¹H NMR (400 MHz, CDCl₃), δ ppm: 13.48 (bs, 1H), 11.60 (bs, 1H), 9.02 (s, 1H), 7.76-7.75 (d, J=3.2 Hz, 1H), 6.96 (d, J=3.6 Hz, 1H), 4.52-4.47 (q, J=7.2,7.2,7.2 Hz, 2H), 1.48-1.44 (t, 7.2, 7.2 Hz, 3H). MS(ES) m/z=225.42 [M+H]⁺.

### Example 2: Preparation of ethyl 4-chloro-1H-pyrrolo[2,3-b]pyridine-5-carboxylate (STG-03)

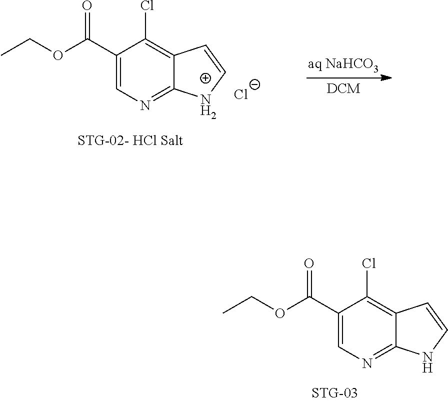

To a solution of ethyl 4-chloro-1H-pyrrolo[2,3-b]pyridine-5-carboxylate hydrochloride (Example 1, STG-01, 7.0 Kg, 1.0 eq.) in DCM (280.0 L, 40.0 vol.) at 25±5° C. and stirred for 10-15 min. The reaction mass was cooled to 15±5° C. then basified with aq. sodium bicarbonate solution (1.5 T, (in water 18.75 vol, 4.6 eq.). The reaction mass temperature was raised to 25±5° C. and stirred for 20-30 min. The layers were separated and extracted the aqueous layer with DCM (35.0 L, 5.0 vol.). The combined organic layer was evaporated under reduced pressure and codistilled with ethanol up to 2-3 vol at 45±5° C. Ethanol (84.0 L, 12.0 vol) was added then heated to 70-80° C. and stirred at 25±5° C. for 30 min. It was concentrated up to 2-3 vol and cooled to 25±5°. There after stirred for 4-6 h, resultant solids were filtered, dried under vacuum at 45±5° C. to obtain ethyl 4-chloro-¹H-pyrrolo[2,3-b]pyridine-5-carboxylate (STG-03) as a pale brown solid (4.49 Kg, 75%). ¹H NMR (400 MHz, DMSO), δ ppm: 12.38 (s, 1H), 8.70 (s, 1H), 7.71-7.70 (t, J=2.8, 3.2 Hz, 1H), 6.66-6.64 (q, J=2.0, 1.2, 2.0 Hz, 1H), 4.38-4.33 (q, J=7.2, 7.2, 6.8 Hz, 2H), 1.37-1.34 (t, J=7.2, 6.8 Hz, 3H). MS(ES) m/z=225.43 [M+H]⁺.

### Example 3: Preparation of Ethyl (R)-4-((1-(tert-butoxycarbonyl)piperidin-3-yl)amino)-1H-pyrrolo[2,3-b]pyridine-5-carboxylate (STG-04)

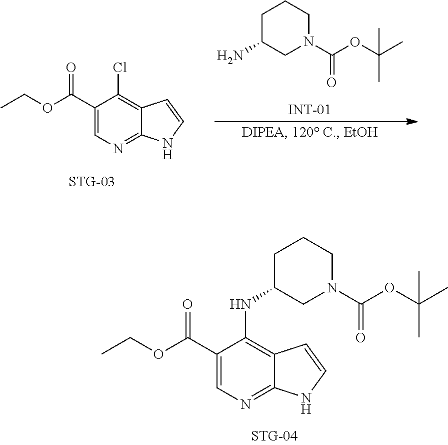 [SMILES: CCOC(=O)c1cnc2[nH]ccc2c1Cl ; CC(C)(C)OC(=O)N1CCC[C@@H](N)C1 ; CCOC(=O)c1cnc2[nH]ccc2c1N[C@@H]1CCCN(C(=O)OC(C)(C)C)C1]

To a stirred suspension of ethyl 4-chloro-1H-pyrrolo[2,3-b]pyridine-5-carboxylate (Example 2, STG-03, 1.25 Kg, 1.0 eq.) in Ethanol (4.0 vol.), were added DIPEA (2.15 Kg, 3.0 eq.), and (R)-3-aminopiperidine-1-carboxylate (INT-01, 1.81 Kg, 1.625 eq.) at 30±5° C. then allowed mass to heat to 120 ° C. in autoclave and maintained at the same temperature for 24-48 h. After completion of reaction (monitored by HPLC), cool to 30±5° C., unload the mass and add Ethanol (5.0 L, 4.0 vol.). The resulting mass was heated to 60±5° C. , water (25.0 L, 20.0 vol.) was added slowly at same temperature. Then cooled to 30±5° C., resulting solids stirred for 1-2 h and filtered, dried under vacuum to obtain the ethyl (R)-4-((1-(tert-butoxycarbonyl)piperidin-3-yl)amino)-1H-pyrrolo[2,3-b]pyridine-5-carboxylate (STG-04) as an off white solid (1.91 Kg, 90.0%). ¹H NMR (400 MHz, DMSO), δ ppm: 11.69 (s, 1H), 8.96-8.94 (d, 7.6 Hz, 1H), 8.56 (s, 1H), 7.21-7.20 (q, J=2.8, 0.4, 2.8 Hz, 1H), 6.63 (bs, 1H), 4.28-4.24 (m, 2H), 4.25-4.16 (m, 1H), 3.8-3.2 (m, 4H), 2.02-1.96 (m, 1H), 1.71-1.69 (bs, 2H), 1.55 (bs, 1H), 1.50-1.32 (m, 3 H), 1.30-1.26 (t, J=7.2, 7.2 Hz, 3 H), 1.25-1.16 (m, 6H). MS(ES) m/z=389.82 [M+H]⁺.

### Example 4: Preparation of Ethyl (R)-4-(piperidin-3-ylamino)-1H-pyrrolo[2,3-b]pyridine-5-carboxylate dihydrochloride salt (STG-05)

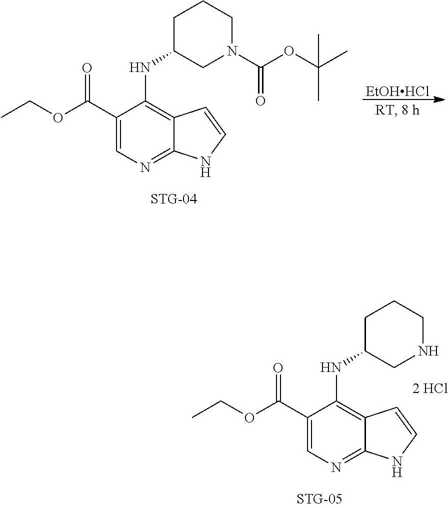

To a stirred suspension of ethyl (R)-4-((1-(tert-butoxycarbonyl)piperidin-3-yl)amino)-1H-pyrrolo[2,3-b]pyridine-5-carboxylate (Example 3, STG-04, 1.9 Kg, 1.0 eq.) in EtOH (9.5 L, 5.0 Vol.) was added EtOH·HCl (9.5 L, 5.0 M, (20%), 5.0 Vol.) slowly at 15±5° C. The reaction mixture was stirred at 30±5° C. for 12-16 h. Progress of the reaction was monitored by HPLC, diluted with MTBE (9.5 L, 5.0 Vol.) and stirred for 4-6 h. The resulting solids were filtered, washed with MTBE (3.8 L, 2.0 vol.) and dried under vacuum to obtain the Ethyl (R)-4-(piperidin-3-ylamino)-1H-pyrrolo[2,3-b]pyridine-5-carboxylate dihydrochloride salt (STG-05) as an off white solid (1.64 Kg, 93.0%). ¹H NMR (400 MHz, DMSO), δ ppm: 12.85 (s, 1H), 10.12-10.10 (m, 1H), 9.48-9.46 (d, J=8.4 Hz, 2H), 7.45-7.44 (q, J=1.6, 1.6, 1.6 Hz, 1H), 7.37-7.36 (d, J=3.2 Hz, 1H), 4.69-4.64 (m,1H), 4.38-4.33 (q, J=7.2, 6.8, 7.2 Hz, 2H),3.45-3.41 (m, 1H), 3.27-3.24 (m, 1H), 3.08-3.00 (m, 1H), 2.88-2.86 (m, 1H), 2.19-2.17 (m, 1H), 2.03-1.93 (m, 2H), 1.90-1.76 (m, 1H), 1.38-1.34 (t, J=7.2, 7.2 Hz, 3H). MS(ES) m/z=289.53 [M+H]⁺.

### Example 5: Preparation of Ethyl (R)-4-41-(2-cyanoacetyl)piperidin-3-yl)amino)-1H-pyrrolo[2,3-b]pyridine-5-carboxylate (STG-06)

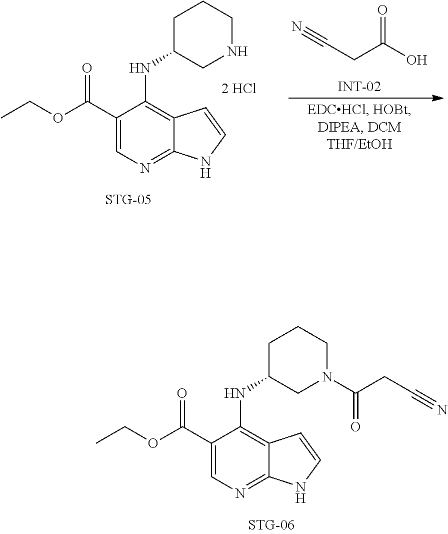 [SMILES: CCOC(=O)c1cnc2[nH]ccc2c1N[C@@H]1CCCNC1 ; N#CCC(=O)O ; CCOC(=O)c1cnc2[nH]ccc2c1N[C@@H]1CCCN(C(=O)CC#N)C1]

To a stirred solution of Cyanoaceticacid (0.829 Kg, 2.5 eq.) and Ethyl (R)-4-(piperidin-3-ylamino)-1H-pyrrolo[2,3-b]pyridine-5-carboxylate dihydrochloride salt (Example 4, 1.4 Kg, 1.0 eq.) in DCM (21.0 L, 15.0 vol.) were added DIPEA (1.75 Kg, 3.5 eq.) slowly, followed by HOBt (1.31 Kg, 2.5 eq.) and finally EDC·HCl (1.86 Kg, 2.5 eq.) was added at 10±5° C. The resulting reaction mass was stirred at same temperature for 30-45 min. Progress of the reaction was monitored by HPLC. Water (7.0 L, 5.0 vol.) was added to the reaction mass and stirred for 10-15 min. The layers were separated. The aqueous layer was extracted with DCM (3×8.0 vol.) and combined the organic layers. The organic layer was washed with 5% aq. sodium bicarbonate solution (3×5.0 vol.) followed by 10% sodium chloride solution (7.0 L, 5.0 vol.). The organic layer was diluted with DCM (14.0 L, 10.0 vol.). SC-40 carbon (0.28 Kg, 0.2 T) and silica gel (0.28 Kg, 0.2 T) were added to the organic layer. The resulting solution was stirred for 1 h and filtered. The filtrate was evaporated under reduced pressure up to 3.0 Vol, co distilled with ethanol (3×5.0 vol.) and concentrated up to 3.0 Vol. Ethanol (2.8 L, 2.0 vol.) was added to the resulting mass and stirred for 4-5 h. The precipitated product was filtered to obtain the Ethyl (R)-4-((1-(2-cyanoacetyl)piperidin-3-yl)amino)-1H-pyrrolo[2,3-b]pyridine-5-carboxylate as a crude(A, ˜507 g.). The obtained solid was added into mixture of THF (A×17.6 vol.) and ethanol (A×4.4 vol.) at 25±5° C. and stirred for 10-15 min. The resulting suspension was heated to 60±5° C. Charcoal was (Eno-Pc, A×0.2T) added at same temperature and stirred for 1 h. Reaction mass was cooled to 55±5° C., filtered through hyflow bed (A×0.1 T) , the bed was washed with mixture of hot (55±5° C). THF(A×8.8 vol.) and Ethanol(A×2.2 vol.) and suck dry the bed. The filtrate was evaporated under reduced pressure up to 3.0 Vol, co distilled with ethanol (3×5.0 vol.) and concentrated up to 3.0 Vol. Ethanol (A×2.0 vol.) was added to the resulting solids and stirred for 4-6 h at 30±5° C. The mass obtained was filtered, dried under vacuum to obtain the Ethyl (R)-4-((1-(2-cyanoacetyl)piperidin-3-yl)amino)-1H-pyrrolo[2,3 -b]pyridine-5 -carboxylate (STG-06) as a pale brown solid (750.0 g, 54.3%). ¹H NMR (400 MHz, DMSO), δ ppm: 11.72 (bs, 1H), 8.89-8.82 (dd, J=8.4, 9.2, 8.0 Hz, 1H), 8.57-8.56 (d, J=3.6 Hz, 1H), 7.23-7.22 (t, J=3.2, 2.8 Hz, 1H), 6.71-6.69 (q, J=2.0, 2.0, 1.6 Hz, 1H), 4.35-4.15 (q, J=7.2,7.2, 6.8, Hz, 3H), 4.11-3.47 (m, 4H), 3.40-3.12 (m, 2H), 2.18-2.02 (m, 1H), 1.82-1.50 (m, 3H), 1.34-1.30 (t, J=7.2, 7.2 Hz, 3H). MS(ES) m/z=356.70 [M+H]⁺.

### Example 6: Preparation of Ethyl (R)-4-((1-(2-cyanoacetyl)piperidin-3-yl)amino)-1H-pyrrolo[2,3-b]pyridine-5-carboxylate (Compound 1)

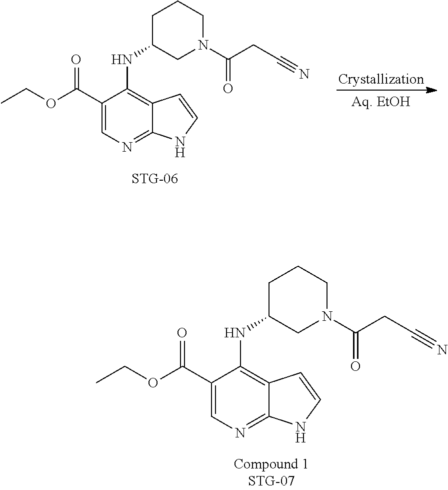 [SMILES: CCOC(=O)c1cnc2[nH]ccc2c1N[C@@H]1CCCN(C(=O)CC#N)C1 ; CCOC(=O)c1cnc2[nH]ccc2c1N[C@@H]1CCCN(C(=O)CC#N)C1]

A suspension of Ethyl (R)-4-((1-(2-cyanoacetyl)piperidin-3-yl)amino)-1H-pyrrolo[2,3-b]pyridine-5-carboxylate (Example 5, STG-06, 650.0 g, 1.0 eq.) in Aq. ethanol (9.1 L, 20%, 14.0 vol.)) was stirred at 70-75° C. for 1-2 h (clear solution observed). The resulting reaction mass was filtered through 0.2 micron, washed with Aq. ethanol (1.3 L, 20%, 2.0 vol.) and filtrate was cooled to 25±5° C., again it was cooled to 10±5° C. and stirred for 4-6 h. The resulting solids were filtered, washed with Ethanol (0.65 L, 1.0 vol.) and dried under vacuum to obtain the Ethyl (R)-4-((1-(2-cyanoacetyl)piperidin-3-yl)amino)-1H-pyrrolo[2,3-b]pyridine-5-carboxylate (STG-07) as a pure product (570.0 g, 87.69%). ¹H NMR (400 MHz, DMSO), δ ppm: 11.72 (bs, 1H), 8.89-8.82 (dd, J=8.4, 9.2, 8.0 Hz, 1H), 8.57-8.56 (d, J=3.6 Hz, 1H), 7.23-7.22 (t, J=3.2, 2.8 Hz, 1H), 6.71-6.69 (q, J=2.0, 2.0, 1.6 Hz, 1H), 4.35-4.15 (q, J=7.2,7.2, 6.8, Hz, 3H), 4.11-3.47 (m, 4H), 3.40-3.12 (m, 2H), 2.18-2.02 (m, 1H), 1.82-1.50 (m, 3H), 1.34-1.30 (t, J=7.2, 7.2 Hz, 3H). MS(ES) m/z=356.70 [M+H]⁺.

### Example 7: X-Ray Structure Determination and PXRD Analysis:

Integration of the data collected from the single crystal of Compound 1 yielded a total of 54,767 reflections to a maximum θ angle of 71.09°, of which 3,222 were independent and 3,213 were greater than 2σ(F2). The final cell constants of a=5.0232 Å, b=9.3308 Å, and c=37.863 Å with a volume of 1774.7 Å³ are based on the refinement of the xyz-centroids of reflections above 20σ(I). The calculated density was 1.330 g/cm³.

Table 1 below provides those peaks detected as significant as well as the corresponding d-spacing for both experimental data as well as simulated data calculated from measurements of a single crystal.

| | |
|---|---|
| Experimental Data | Simulated Data |

| °2Θ | d-spacing (Å) | °2Θ | d-spacing (Å) |
|---|---|---|---|
| 4.67 | 18.92 | 4.67 | 18.92 |
| 9.33 | 9.48 | 9.34 | 9.47 |
| 9.55 | 9.26 | 9.47 | 9.34 |
| 9.69 | 9.13 | 9.76 | 9.06 |
| 10.50 | 8.42 | 10.56 | 8.38 |
| 11.65 | 7.60 | 11.70 | 7.56 |
| 11.73 | 7.54 | 11.82 | 7.49 |
| 14.01 | 6.32 | 14.02 | 6.32 |
| 17.63 | 5.03 | 17.80 | 4.98 |
| | | 17.66 | 5.02 |
| 18.73 | 4.74 | 18.74 | 4.73 |
| 18.86 | 4.70 | 19.00 | 4.67 |
| 19.78 | 4.49 | 20.00 | 4.44 |
| 19.86 | 4.47 | 20.06 | 4.43 |
| 19.95 | 4.45 | 20.20 | 4.40 |
| 20.04 | 4.43 | 20.29 | 4.38 |
| 20.42 | 4.35 | 20.60 | 4.31 |
| 21.00 | 4.23 | 21.22 | 4.19 |
| 21.11 | 4.21 | 21.27 | 4.18 |
| 23.13 | 3.84 | 23.28 | 3.82 |
| 23.59 | 3.77 | 23.70 | 3.75 |
| 24.03 | 3.70 | 24.16 | 3.68 |
| 24.40 | 3.65 | 24.56 | 3.62 |
| 25.85 | 3.45 | 26.02 | 342 |
| | | 26.05 | 3.42 |
| 26.28 | 3.39 | 26.48 | 3.37 |
| 27.46 | 3.25 | 27.59 | 3.23 |
| | | 27.65 | 3.23 |
| | | 27.72 | 3.22 |

Table 2 below provides the and their intensities of the FT-Raman spectrum.

| Peak (cm⁻¹) | Intensity |
|---|---|
| 413.7 | 6.827 |
| 441.3 | 5.996 |
| 596.3 | 8.718 |
| 663.5 | 19.862 |
| 735.9 | 13.845 |
| 784.2 | 12.47 |
| 871.3 | 16.713 |
| 906.5 | 10.313 |
| 933.1 | 6.477 |
| 974.1 | 8.384 |
| 1045.4 | 5.347 |
| 1097.1 | 6.355 |
| 1114.5 | 17.727 |
| 1146.7 | 9.816 |
| 1226.1 | 8.747 |
| 1253.9 | 20.742 |
| 1281.5 | 11.656 |
| 1307.1 | 8.514 |
| 1343.8 | 28.008 |
| 1372.7 | 31.867 |
| 1394.2 | 8.481 |
| 1440.5 | 13.321 |
| 1452.4 | 14.345 |
| 1499.7 | 53.449 |
| 1581.5 | 7.052 |
| 1662.5 | 27.729 |
| 2258.4 | 10.015 |
| 2873.3 | 8.525 |
| 2921.6 | 17.799 |
| 2965 | 17.47 |
| 3139.5 | 7.136 |

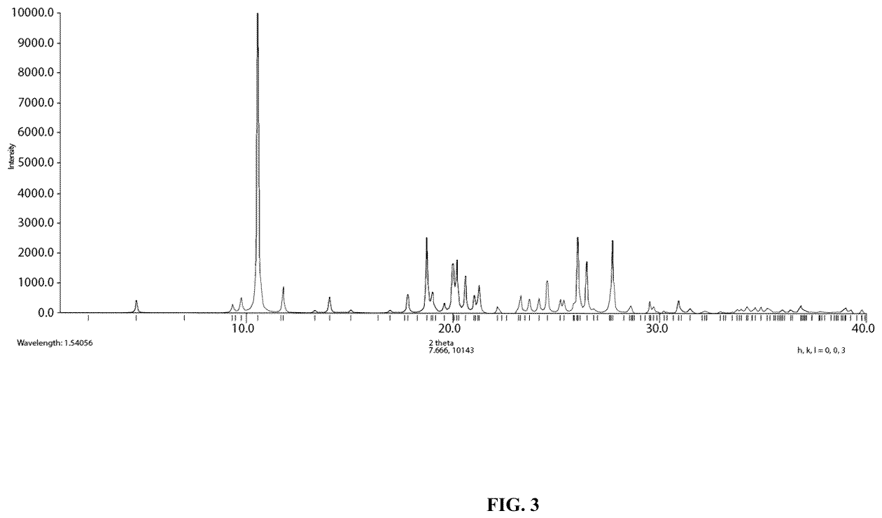

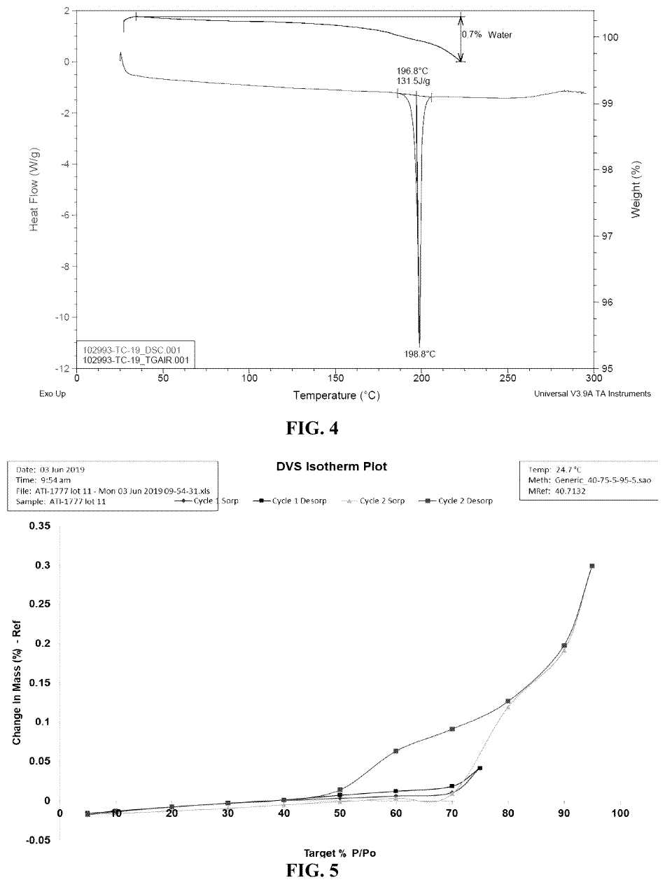

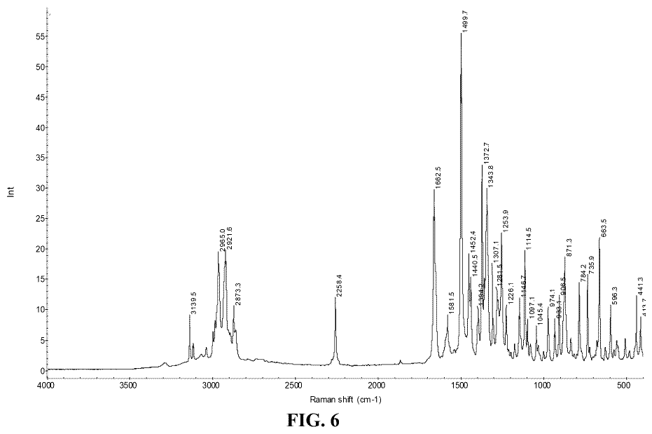

FIG. 2 and FIG. 3 provide representative PXRD patterns of the experimental data collected on a bulk sample of Compound 1 and a simulated PXRD pattern calculated using measurements from a single Compound 1 crystal. FIG. 4 provide representative TGA-IR and DSC curves for Compound 1, illustrating a 0.7 wt. % loss of water from 35° C. to 225° C. and a sharp endotherm with an onset at 196.8° C., respectively. FIG. 5 provides the results of DVS analysis on a batch of Compound 1, revealing that the bulk material has low moisture uptake. FIG. 6 provides a representative FT-Raman spectrum of Compound 1.

Although the present invention has been described in considerable detail with reference to certain preferred embodiments thereof, other versions are possible. Therefore the spirit and scope of the appended claims should not be limited to the description and the preferred versions contained within this specification.

1. Crystalline polymorph Form A of Compound 1 (ethyl (R)-4-((1-(2-cyanoacetyl)piperidin-3-yl)amino)-1H-pyrrolo[2,3-b]pyridine-5-carboxylate) characterized by a PXRD pattern comprising a significant peak at a 2θ angle of about 10.50°.

2. The crystalline polymorph of claim 1, wherein the PXRD pattern further comprises a significant peak at 2θ angle of about 18.86°.

3. The crystalline polymorph of claim 1, wherein the PXRD pattern further comprises significant peaks at 2θ angles of about 9.69°, about 14.01°, and about 25.85°.

4. The crystalline polymorph of claim 1, wherein the PXRD pattern further comprises significant peaks at 2θ of about 4.67°, about 9.33°, about 9.55°, and about 27.46°.

5. Crystalline polymorph Form A of Compound 1 (ethyl (R)-4-((1-(2-cyanoacetyl)piperidin-3-yl)amino)-1H-pyrrolo[2,3-b]pyridine-5-carboxylate) characterized by a FT-Raman spectra comprising a significant peak at about 1499.7 cm⁻¹.

6. The crystalline polymorph of claim 5, wherein the FT-Raman spectra further comprises a significant peak at about 31.867 cm⁻¹.

7. The crystalline polymorph of claim 5, wherein the FT-Raman spectra further comprises significant peaks at about 28.008 cm⁻¹, about 27.729 cm⁻¹, about 20.742 cm⁻¹, and about 19.862 cm⁻¹.

8. The crystalline polymorph of claim 5, wherein the FT-Raman spectra further comprises significant peaks at about 17.799 cm⁻¹, about 17.727 cm⁻¹, about 17.47 cm⁻¹, and about 16.713 cm⁻¹.

9. Crystalline polymorph Form A of Compound 1 (ethyl (R)-4-((1-(2-cyanoacetyl)piperidin-3-yl)amino)-1H-pyrrolo[2,3-b]pyridine-5-carboxylate) characterized by a PXRD pattern comprising a significant peak at a 2θ angle of about 10.50° and characterized by a FT-Raman spectra comprising a significant peak at about 1499.7 cm⁻¹.

10. The crystalline polymorph of claim 9, wherein the PXRD pattern further comprises a significant peak at 2θ angle of about 18.86° and wherein the FT-Raman spectra further comprises a significant peak at about 31.867 cm⁻¹.

11. The crystalline polymorph of claim 9, wherein the PXRD pattern further comprises significant peaks at 2θ angles of about 9.69°, about 14.01°, and about 25.85° and wherein the FT-Raman spectra further comprises a significant peak at about 28.008 cm⁻¹, about 27.729 cm⁻¹, about 20.742 cm⁻¹, and about 19.862 cm⁻¹.

12. The crystalline polymorph of claim 9, wherein the PXRD pattern further comprises significant peaks at 2θ angles of about 4.67°, about 9.33°, about 9.55°, and about 27.46° and wherein the FT-Raman spectra further comprises a significant peak at about 17.799 cm⁻¹, about 17.727 cm⁻¹, about 17.47 cm⁻¹, and about 16.713 cm⁻¹.

13. The crystalline polymorph of claim 1, further characterized by a PXRD pattern substantially as shown in FIG. 2 or FIG. 3.

14. The crystalline polymorph of claim 1, further characterized by a DSC thermograms exhibiting an endotherm at about 196.8° C.

15. The crystalline polymorph of claim 1, further characterized by a water loss as measured by thermogravimetric analysis of about 0.7 wt. %.

16. The crystalline polymorph of claim 1, further characterized by a FT-Raman spectra substantially as shown in FIG. 6.

17. A method of preparing crystalline polymorph Form A of Compound 1 (ethyl (R)-4-((1-(2-cyanoacetyl)piperidin-3-yl)amino)-1H-pyrrolo[2,3-b]pyridine-5-carboxylate) comprising:
combining Compound 1 and an alcohol to form a reaction mixture;
heating the reaction mixture to a temperature sufficient to form a clear solution; and cooling the clear solution to a temperature of about 10° C. to about 15° C. to yield crystalline polymorph Form A of Compound 1 as a solid precipitate.

18.-25. (canceled)

26. A process for the preparation of Compound 1 having the structure:
comprising the steps of: 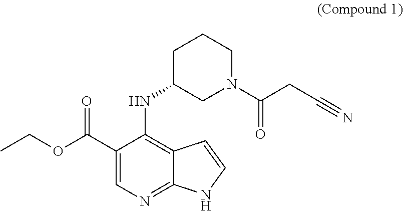 [SMILES: CCOC(=O)c1cnc2[nH]ccc2c1N[C@@H]1CCCN(C(=O)CC#N)C1]
(a) contacting the compound 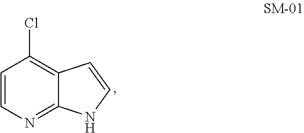
with the compound 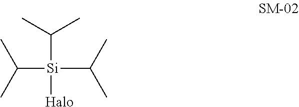
in the presence of a base to form the compound 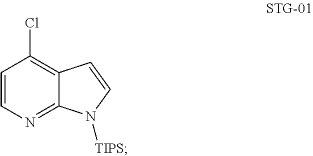
and
(b) converting STG-01 to Compound 1.

27.-48. (canceled)
